(19) United States
(12) Patent Application Publication
(10) Pub. No.: US 2026/0327120 A1
(43) Pub. Date: Oct. 22, 2026

(54) MODULAR SELF-BALLASTING ANAEROBIC DIGESTER APPARATUS

(71) Applicant: R5 Energy Limited, West Byfleet (GB)

(72) Inventor: Waqas Ahmad, Woking (GB)

(21) Appl. No.: 19/646,744

(22) Filed: Apr. 14, 2026

(30) Foreign Application Priority Data
Apr. 16, 2025 (GB) 2505743.1

(51) Int. Cl. C02F 11/04; C05F 17/10; C05F 17/993; F28D 15/02

(52) U.S. Cl. CPC C02F 11/04; C05F 17/10; C05F 17/993; F28D 15/02

(57) ABSTRACT

An apparatus and method for a modular self-ballasting anaerobic digestion apparatus are described. In one aspect, A modular self-ballasting anaerobic digester apparatus includes gas-filled modules mechanically connected to form a container structure with a load-bearing space, roof, walls, and floor. A composite tanking apparatus comprises multiple layers, including an outer blast mitigation layer, gas-absorbent insulation layer, heat transfer layer, and digestion layer. Heat pipes sealed within the heat transfer layer transfer thermal energy between a heat source and the digestion layer. A carbon capture apparatus with a thermal storage tank and carbon capture heat pipes extracts thermal energy from carbon dioxide and transfers it to a thermal storage medium. Metering valves control the release of cooled carbon dioxide into the digestion layer. A deterministic control system monitors time, temperature, and liquid level parameters within the digestion layer, executing deterministic rules for controlling the anaerobic digester process.

### FIELD

Various embodiments of the disclosure relate to an anaerobic digester apparatus and, more particularly, to a modular, self-ballasting anaerobic digester with integrated carbon capture and heat management capabilities.

### BACKGROUND

Anaerobic digester apparatuses are typically used to process organic waste materials and produce biogas as a renewable energy source. Traditional anaerobic digester apparatuses typically may include large, fixed installations that require significant infrastructure and are challenging to scale or relocate. Further, traditional anaerobic digesters may face operational challenges, including uneven temperature distribution, inefficient heat management, and limited ability to capture and utilize carbon dioxide produced during the digestion process. Traditional anaerobic digesters may frequently rely on concrete or steel tanks that necessitate extensive site preparation, including excavation and foundation work. Such an approach may limit flexibility in terms of scaling up apparatus capacity and determining the location of installation. Further, the traditional anaerobic digesters typically exhibit thermal stratification and inadequate heat distribution throughout the reactor volume, resulting in suboptimal microbial activity, reduced metabolic efficiency, and diminished biogas production rates.

Most traditional anaerobic digesters may release carbon dioxide, a byproduct of the digestion process, directly into the atmosphere. This practice not only represents a missed opportunity for carbon capture and utilization but also raises environmental concerns. Additionally, traditional anaerobic digesters typically lack effective integration with renewable energy sources such as solar power, which could otherwise help offset their operational energy requirements and improve overall sustainability. Heat management poses another challenge for traditional anaerobic digesters. Inefficient heat transfer mechanisms may result in temperature fluctuations within the digester, negatively impacting microbial activity and overall apparatus performance. Many traditional anaerobic digesters are not only inefficient but also fail to effectively distribute and retain heat, leading to increased energy consumption to maintain optimal conditions. The fixed nature of conventional installations has hindered the scalability of anaerobic digester technology. Adapting an apparatus capacity to change waste volumes or relocating digesters to new sites typically involves considerable time, effort, and cost. This lack of modularity and portability limits the widespread adoption of anaerobic digesters, particularly in scenarios where waste production volumes may vary or temporary installations are desired.

Therefore, providing an anaerobic digester that may include modular construction, self-ballasting capabilities, efficient heat management apparatus, integrated carbon capture management, and a scalable design that may be easily installed or relocated may be challenging.

### SUMMARY

Provided is a modular self-ballasting anaerobic digestion apparatus as claimed in claim 1.

Also provided is a method as claimed in claim 17.

In an embodiment, the apparatus and method provide a composite tanking apparatus or a composite tanking system formed within a modular structure of a mechanically connected multiple gas-filled modules. The modular structure provides a load-bearing space, a roof, walls, and a floor. The gas-filled modules are configured to provide the modular structure, eliminating the need for external foundations or support structures. In an embodiment, the apparatus and method provide a composite tank apparatus, including, for example, an interceptor tank (also referred to as a pre-treatment tank or a pre-treatment apparatus) and/or a digester tank. The composite tanking apparatus may be formed of or include multiple layers and may be disposed within the modular structure. The layered design enables a protection that enhances both operational efficiency and safety by containing the anaerobic digestion process within a multi-barrier environment.

In an embodiment, the apparatus and method provide an outer blast mitigation layer comprising a woven fabric, including ceramic spheres. This blast mitigation layer provides absorption and may disperse energy, reduce the risks associated with biogas production, and maintain the structural integrity of the apparatus. In an embodiment, the apparatus and method provide a gas-absorbent insulation layer in the outer blast mitigation layer. This gas-absorbent insulation layer may be operable to capture any gases and provide thermal insulation, which stabilizes the interior environment and improves the operational efficiency of the digestion process.

In an embodiment, the apparatus and method provide a heat transfer layer, including a sealed bladder in the gas-absorbent insulation layer. The sealed heat transfer bladder creates a dedicated thermal management zone that maintains optimal temperature conditions for microbial activity without introducing contamination risks. In an embodiment, the apparatus and method provide a digestion layer, including a flexible bladder (also referred to as digester bladder or digester layer) for containing organic matter positioned on top of the heat transfer layer. The digestion bladder may be operable to accommodate volume changes during the digestion process and maintain appropriate temperatures by the underlying heat transfer layer. In an embodiment, the apparatus and method provide heat pipes sealed within the heat transfer layer. The heat pipes may be adapted to transfer thermal energy between a heat source and the digestion layer using a heat transfer medium. The sealed heat pipes transfer thermal energy without direct contact between heating elements and the digestion contents, eliminating fouling issues and providing even temperature distribution.

In an embodiment, the apparatus and method provide a carbon capture apparatus or system operably connected to the digestion layer. The carbon capture apparatus enables the modular self-ballasting anaerobic digestor apparatus to reduce greenhouse gas emissions and improve biogas production through controlled carbon dioxide reuse. In an embodiment, the apparatus and method provide a thermal storage tank. The thermal storage tank functions as a reservoir for captured thermal energy, enabling efficient energy management and reducing parasitic energy losses common in conventional anaerobic digestion apparatuses. In an embodiment, the apparatus and method provide carbon capture heat pipes in the thermal storage tank, wherein the carbon capture heat pipes are operable to extract thermal energy from carbon dioxide and transfer the thermal energy to a thermal storage medium within the thermal storage tank. The heat pipes cool carbon dioxide by extracting thermal energy, which results in reduced gas volume and improved storage efficiency without requiring cryogenic or compression technologies.

In an embodiment, the apparatus and method provide a metering valve configured to control a release of cooled carbon dioxide into the digestion layer and a pre-treatment apparatus. The metering valve enables control of carbon dioxide introduction into the digestion process, optimizing biogas production based on real-time process conditions. In an embodiment, the apparatus and method provide a deterministic control system that may be operably connected to the heat pipes and the carbon capture apparatus. The deterministic control system may include sensors configured to monitor time, temperature, and liquid level parameters within the digestion layer. The deterministic control system may be operable to create a deterministic operational environment that prevents failure conditions rather than merely responding to them, thereby increasing operational reliability.

In an embodiment, the apparatus and method provide the deterministic control system or apparatus that may be configured to execute deterministic rules. The deterministic control system may execute operations to apply predefined operational parameters that optimize digestion performance based on feedstock characteristics. In an embodiment, the apparatus and method provide gas-filled modules connected using torque tubes that are adapted to provide structural elements and guttering for liquid collection. The dual-function torque tubes create an integrated structure that enhances the overall efficiency by collecting water and maintaining structural integrity.

In an embodiment, the apparatus and method provide guttering positioned beneath the solar panels that may be operable to collect water runoff and direct the collected water to the digestion layer. The water collection mechanism through the guttering may also capture atmospheric moisture for integration into the digestion process, reducing external water requirements. In an embodiment, the apparatus and method provide gas entrapped within the gas-filled modules, which provides buoyancy to create a load-bearing ballast. The entrapped gas creates the self-ballasting apparatus that eliminates the need for conventional foundations, ground anchoring, or excavation work typically required for digesters. In an embodiment, the apparatus and method may provide ceramic spheres in the outer blast mitigation layer that includes an expandable foam coated with a ceramic liner. The expandable foam-filled ceramic spheres may absorb energy through deformation and maintaining containment properties, creating a progressive energy dissipation apparatus.

In an embodiment, the apparatus and method provide gas-filled modules adapted to be mechanically connected to form the container structure with an aspect ratio of 5:1. The specific aspect ratio of 5:1 optimizes structural performance. In an embodiment, the apparatus and method provide a roof that may include mounted solar panels operable to be connected to the heat pipes. The roof-mounted solar panels capture solar energy for direct thermal transfer to the digestion process, reducing external energy requirements. In an embodiment, the apparatus and method provide a carbon capture apparatus or system that further comprises a valve controlled by the deterministic control system to meter carbon dioxide injection into the digestion layer based on the monitored parameters. The controlled carbon dioxide injection enhances methanogenesis in the digestion process, increasing biogas yield compared to conventional apparatuses. In an embodiment, the apparatus and method provide a digestion process that comprises temperature-phased digestion with extended sludge retention. The temperature-phased process with extended retention maximizes organic matter conversion and stabilizes the microbial community, resulting in higher biogas yields.

In an embodiment, the apparatus and method provide a pre-treatment mechanism that is operable to prepare organic matter before transferring the pretreated organic matter to the digestion layer located in the load-bearing space. The pre-treatment mechanism optimizes feedstock characteristics for digestion, reducing inhibition risks and improving overall apparatus performance. In an embodiment, the apparatus and method provide an outer blast mitigation layer that is configured to absorb and reduce blast effects and debris through the expansion of the expandable foam contained within the ceramic spheres. The expansion mechanism creates a controlled deformation zone that absorbs energy and contains materials, thereby enhancing operational safety. In an embodiment, the apparatus and method provide a carbon capture apparatus that is configured to achieve a reduction of at least 50% in carbon dioxide volume through thermal energy extraction via the carbon capture heat pipes. The thermal extraction process substantially reduces carbon dioxide volume without energy-intensive compression, creating an efficient carbon management apparatus.

### BRIEF DESCRIPTION OF THE DRAWINGS

FIG. 1A is an illustration showing an anaerobic digestion system, including a modular structure, a carbon capture system, and a heat transfer system, according to an exemplary embodiment.

FIG. 1B is an illustration showing an isometric view of a modular self-ballasting anaerobic digester apparatus, according to an exemplary embodiment.

FIG. 1C shows a blown-up view of carbon capture components between the interceptor tank and the digester tank, according to an exemplary embodiment.

FIG. 2A is an illustration showing a top view of the modular self-ballasting anaerobic digester apparatus, according to an exemplary embodiment.

FIG. 2B is an illustration showing a side view of the modular self-ballasting anaerobic digester apparatus, according to an exemplary embodiment.

FIG. 2C is an illustration showing an arrangement of gas-filled modules forming the floor module, wall module, and roof module, according to exemplary embodiments.

FIG. 2D is an illustration showing a higher-volume gas-filled module, according to an exemplary embodiment.

FIG. 2E is an illustration showing the mechanical connection and reinforcement of the higher-volume gas-filled modules, according to an exemplary embodiment.

FIG. 2F is an illustration showing a lower-volume gas-filled module, according to an exemplary embodiment.

FIG. 2G is an illustration showing the mechanical connection and reinforcement of the lower-volume gas-filled modules, according to an exemplary embodiment.

FIG. 2H is an illustration showing an exploded view of a composite tanking apparatus, according to an exemplary embodiment.

FIG. 3A is an illustration showing a top view of the arrangement of mechanically connected lower-volume gas-filled modules, according to an exemplary embodiment.

FIG. 3B is an illustration showing a blown-up sectional view of the arrangement of mechanically connected lower-volume gas-filled modules, according to an exemplary embodiment.

FIG. 3C is an illustration showing a side view of the arrangement of mechanically connected lower-volume gas-filled modules, according to an exemplary embodiment.

FIG. 3D is an illustration showing a blown-up sectional view of the arrangement of mechanically connected lower-volume gas-filled modules, according to an exemplary embodiment.

FIG. 3E is an illustration showing a top view of the arrangement of mechanically connected higher-volume gas-filled modules, according to an exemplary embodiment.

FIG. 3F is an illustration showing a blown-up sectional view of the arrangement of mechanically connected higher-volume gas-filled modules, according to an exemplary embodiment.

FIG. 3G is an illustration showing a side view of the arrangement of mechanically connected higher-volume gas-filled modules, according to an exemplary embodiment.

FIG. 3H is an illustration showing a blown-up sectional view of the arrangement of mechanically connected higher-volume gas-filled modules, according to an exemplary embodiment.

FIG. 4A is an illustration showing the structure of a gas-filled module, according to an exemplary embodiment.

FIG. 4B is an illustration showing a blown-up sectional view of the gas-filled module according to an exemplary embodiment.

FIG. 5A is an illustration showing a heat pipe that includes a medium to emit, absorb, or adsorb heat and transfer the heat, according to an exemplary embodiment.

FIG. 5B is an illustration showing heat transfer components, according to an exemplary embodiment.

FIG. 5C is an illustration showing heat transfer components, according to an exemplary embodiment.

FIGS. 6A and 6B are illustrations showing a carbon capture apparatus that includes multiple interconnected components, according to exemplary embodiments.

FIG. 6C and FIG. 6D are illustrations showing a sectional view of the carbon capture apparatus, according to exemplary embodiments.

FIG. 7 is an illustration showing an isometric view of a modular self-ballasting anaerobic digester apparatus, showing an arrangement of multiple layers, according to an exemplary embodiment.

FIG. 8 illustrates a process flow diagram of the modular self-ballasting anaerobic digester apparatus, according to an exemplary embodiment.

FIG. 9 is a flow chart illustrating a process for operating a modular self-ballasting anaerobic digestor apparatus, according to an exemplary embodiment.

### DETAILED DESCRIPTION

The present disclosure relates to a modular self-ballasting anaerobic digester apparatus for processing organic waste and capturing biogas. The modular self-ballasting anaerobic digester apparatus may herein also be interchangeably referred to as an apparatus or an anaerobic digester apparatus or anaerobic digestion system or anerobic digestion apparatus in the subject specification. The apparatus may be deployed or installed in multiple locations without requiring extensive site preparation or permanent foundations.

In an embodiment, the following terms may be used in the subject specification and may be described as follows. An anaerobic digester may correspond to a series of biological processes in which microorganisms break down biodegradable material in the absence of oxygen that may be used for waste management and biogas production. A biogas may correspond to a mixture of gases, primarily methane and carbon dioxide, that the anaerobic digestion of organic matter may produce. A heat pipe may correspond to a heat transfer device that may combine the principles of thermal conductivity and phase transition to transfer heat between two interfaces. A thermosyphon may correspond to a mechanism of passive heat exchange based on natural convection that may circulate a fluid without the necessity of a mechanical pump. A carbon capture mechanism may correspond to a mechanism or a process of capturing carbon dioxide (CO₂) emissions from sources like fossil fuel power plants and either reusing or storing it so that it does not enter the atmosphere. A deterministic control system is also referred to as a control system where the output is determined by the apparatus's parameters and the initial conditions without any random variation.

The present disclosure addresses the technical problem of creating an efficient, mobile, and self-contained anaerobic digester apparatus that can be deployed in various environments without extensive site preparation. The technical solution includes providing a modular design of an anaerobic digester apparatus that uses gas-filled modules that may be adapted for structural support and ballast combined with a multi-layered composite tanking apparatus and advanced thermal management using heat pipes. The apparatus described in the subject specification enables the creation of a versatile and efficient organic waste processing apparatus with integrated carbon capture capabilities.

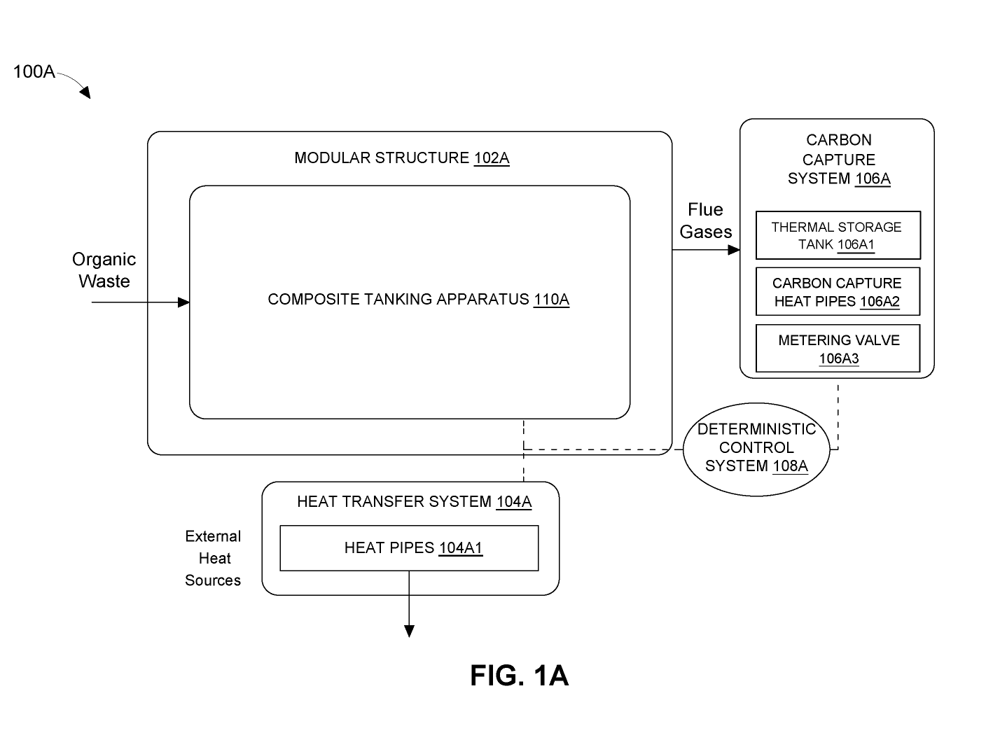

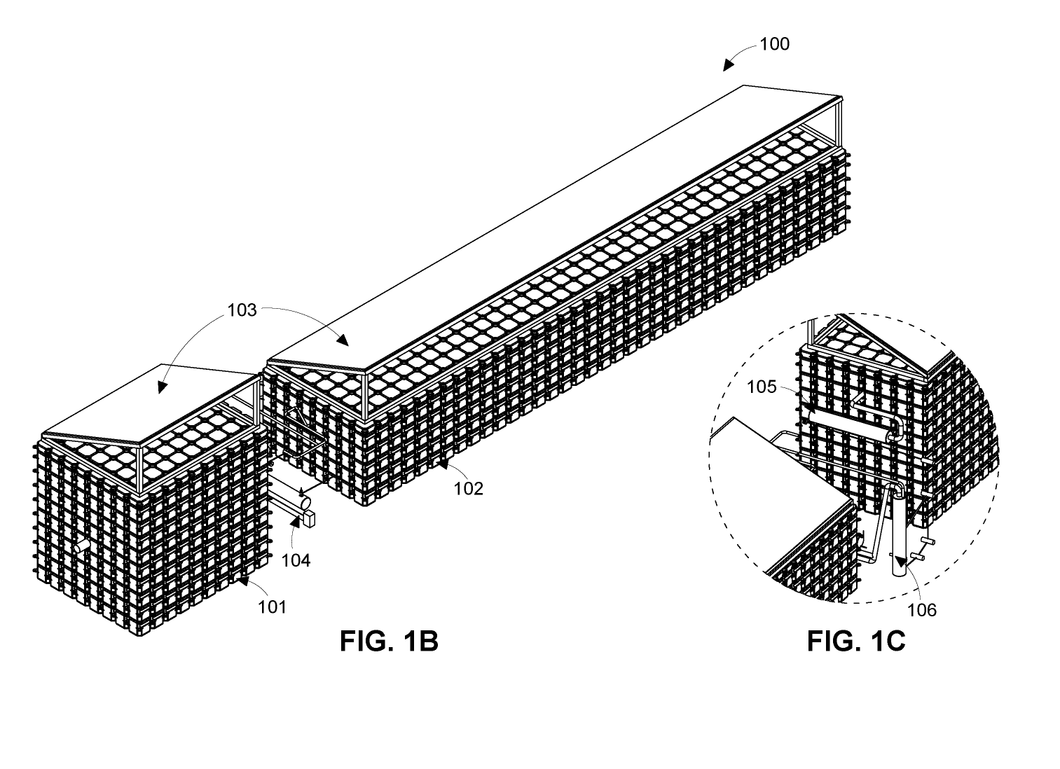

FIG. 1A is an illustration showing a modular self-ballasting anaerobic digestion system, including a modular structure, a carbon capture system, and a heat transfer system, according to an exemplary embodiment. In an embodiment, FIG. 1A shows the modular self-ballasting anaerobic digestion system 100A that may facilitate or be used to efficiently treat organic waste through an anaerobic digestion process or mechanism. The modular self-ballasting anaerobic digestion system 100A may include an integration of multiple components into a modular and structured design that may be deployed or installed to operate on land or water without requiring traditional foundations, piling, or ground screws. The modular self-ballasting anaerobic digestion system 100A’s self-ballasting capability may be achieved through the use of gas-filled modules. The heat transfer system 104A, a carbon capture system 106A, and a deterministic control system 108A may be housed within a modular self-ballasting anaerobic digestion system 100A, forming a composite tanking apparatus 110A.

In operation, the modular self-ballasting anaerobic digestion system 100A, as shown in FIG. 1A may provide a sustainable solution for organic waste management. The modular self-ballasting anaerobic digestion system’s 100A integrated design, including the modular structure 102A, the heat transfer system 104A with the heat pipes 104A1, the carbon capture system 106A including the thermal storage tank 106A1 and carbon capture heat pipes 106A2, the deterministic control system 108A, and the composite tanking apparatus 110A with a gas-absorbent insulation layer (not shown) and a digestion bladder (not shown), enable efficient operation while minimizing environmental impact. The flue gas, external heat sources, etc., indicate the modular self-ballasting anerobic digestion system’s reliance on and interaction with external resources and outputs having applications in multiple scenarios.

In an embodiment, the modular structure 102A may form the foundational framework of the modular self-ballasting anaerobic digestion system 100A. The modular structure 102A may be formed or constructed using multiple gas-filled modules that may form a load-bearing space, a roof, walls, and a floor. The gas-filled modules may be mechanically connected using torque tubes (not shown) that may be operable to provide structural support and guttering. The guttering may be available in 90-degree, 180-degree, and 45-degree hollow configurations. In an embodiment, the torque tubes may be fastened to the gas-filled modules via drilled or glued connecting sections. The outlets within the torque tubes may be operable or function as drain points, allowing water, liquids, or dust to be removed from gas-bearing areas. The gas-filled modules may be filled with air or other gases and may provide buoyancy and act as a load-bearing ballast, eliminating the need for external support. The gas-filled modules may be assembled to any size while maintaining an optimal aspect ratio of 5:1, achieved by attaching them on top, sides, and length using a standardized fastening method, ensuring repeatability and quality in construction.

In an embodiment, within the modular structure 102A, the composite tanking apparatus 110A, also referred to as a composite tanking system, may be included. The composite tanking apparatus 110A may include multiple layers to the digestion process. The mechanism or process may be initiated with organic waste (e.g., shown as entering the composite tanking apparatus 110A) being introduced into, for example, a pre-treatment apparatus that may also be referred to as an interceptor tank (not shown) or a pre-treatment system via pumps and valves. The flow and pre-treatment of this waste are regulated by the deterministic control system 108A, which may implement programmable logic controller (PLC) rules based on time, temperature, and level parameters monitored by sensors. The pre-treatment apparatus may reduce the volume of feedstock using hollow sections and manifolds as contact filters and prepare the organic waste material for subsequent digestion.

In an embodiment, the pre-treated material may then be transferred via pumps and valves into the digestion bladder, where a three-stage temperature-phased digestion process with extended sludge retention occurs. In the first stage, the digestion bladder is heated by a heating bladder (not shown) through the heat transfer system 104A using the heat pipes 104A1. These heat pipes 104A1 may transfer thermal energy from external sources, including flue gas, external heat sources such as solar panels (not shown), and a thermal storage tank (not shown). The heat transfer medium within the heat pipes 104A1 undergoes a phase change to facilitate efficient heat transfer, with temperature-activated valves maintaining the temperature between 32°C and 38°C, the optimal range for mesophilic digestion. The heat loss may be minimized by the insulation layer that enables uniform temperature distribution within the digestion bladder.

In an embodiment, in the second stage, the organic matter is decomposed in an oxygen-free environment, yielding biogas (including carbon dioxide (CO₂)) and liquid fertilizer. A portion of the CO₂ may be captured and cooled by the carbon capture system 106A, which may include a thermal storage tank 106A1, multiple carbon capture heat pipes 106A2, and a metering valve 106A3. In an embodiment, the CO₂ is captured in a tank which has heat pipes to extract the heat and this reduces the temperature and thus the gaseous state thus reducing the velocity and volume to be stored. The system deals with excess CO₂ via a vent which is connected via a piping network to a pre-treatment tank where it mixes with the waste/feedstock from the tank. . The carbon capture system 106A may be designed to capture CO₂ from biogas or flue gases using heat pipes 104A1 arranged in a flat plate configuration with the thermal storage tank. The heat pipes 104A1 may absorb heat from the hot CO₂ gas, reducing its temperature and velocity by approximately 50%. The dew may be collected in a drip tray and removed via a drain valve, while a filter prevents particle entry. The cooled CO₂ is stored in a liquid-filled cavity within CO₂ tanks, with the heat pipes extracting residual heat into the thermal store.

In an embodiment, the third stage may include treating the output material, which is then transferred into a pasteurization tank (not shown) for thermophilic treatment to eliminate living cells, ensuring the effluent’s safety. The deterministic control system 108A may continuously monitor time, level, and temperature, activating bypass valves to prevent overheating or overloading, thereby proactively mitigating system failures. In an embodiment, the heat recapture is efficiently achieved within the modular self-ballasting anaerobic digestion system 100A. The heat from solar panels, which are a part of the heat transfer system 104A, is utilized, with guttering formed by the torque tubes collecting runoff water heated by the panels and directing it into the digester tanks. Similarly, the heat from the flue gas is captured and transferred to the heating bladder via the heat pipes 104A1, thereby reducing parasitic biogas consumption by 30% and enhancing overall efficiency.

In an embodiment, the final effluent produced includes biogas, liquid fertilizer, and digestate. The output volume is increased by 30% compared to conventional digesters, a result of the effective heat recapture and insulation mechanisms. The biogas may be extracted using blowers while liquid fertilizer and digestate are pumped to storage or utilization points.

FIG. 1B is an illustration showing an isometric view of a modular self-ballasting anaerobic digester apparatus 100, according to an exemplary embodiment. The modular self-ballasting anaerobic digester apparatus 100 may include, for example, an interceptor tank 101, a digester tank 102, a solar panel 103, and a heat exchanger 104. In an embodiment, the interceptor tank 101 and the digester tank 102 may be mechanically connected to form a container structure. The container structure may be formed or include a load-bearing space, walls, a floor, and a roof. Solar panels 103 may be mounted on the roof structure of apparatus 100 to capture solar energy. The heat exchanger 104 may be positioned between the interceptor tank 101 and the digester tank 102 to facilitate thermal transfer between apparatus components.

FIG. 1C shows a blown-up view of carbon capture components 105 and 106 between the interceptor tank 101 and the digester tank 102, according to an exemplary embodiment. In an embodiment, the modular design may enable the components of the apparatus 100 to be assembled and disassembled, as well as maintain structural integrity. In an embodiment, the modular self-ballasting anaerobic digester apparatus 100 may maintain an aspect ratio of 5:1 through a modular assembly. For instance, the aspect ratio of 5:1 may correspond to one dimension being five times greater than the other of the gas-filled modules, thereby creating ideal conditions for fluid dynamics within the digestion process. For example, the gas-filled module with dimensions of 5 meters in length and 1 meter in width (creating the 5:1 aspect ratio) may be arranged horizontally within the digestion chamber. When organic slurry flows through this elongated rectangular path, the 5:1 aspect ratio may enable the creation of a flow pattern where the material moves at an optimal velocity, fast enough to prevent the sedimentation of suspended solids but slow enough to allow sufficient contact time between microorganisms and organic matter.

In an embodiment, when designing the heat transfer channels within the sealed bladder system, a 5:1 aspect ratio may maximize the surface area contact and maintain appropriate flow rates. Such an arrangement enables optimal heat distribution throughout the digestion medium. The aspect ratio of 5:1 may enable the heat pipes to effectively transfer thermal energy from various sources (for example, solar panel, waste heat, ground source, etc.) into the digestion chamber without creating temperature gradients that could impair microbial activity. Similarly, the carbon capture apparatus utilizes the 5:1 aspect ratio in its gas flow pathways, where the higher-volume dimension enables sufficient time for gas cooling and volume reduction, and the lower-volume dimension maintains appropriate pressure and flow velocity. For example, in the carbon capture apparatus, a heat exchange chamber may be constructed with dimensions of 2.5 meters in length and 0.5 meters in diameter, maintaining the 5:1 aspect ratio. When hot CO₂-rich flue gas (approximately 200°C) enters this chamber, it travels along the extended 2.5-meter pathway where an array of heat pipes is positioned at regular intervals. This elongated dimension provides sufficient residence time (approximately 3-4 seconds) for the gas to interact with the heat transfer surfaces, allowing thermal energy to be efficiently extracted and stored. As the gas traverses this extended pathway, its temperature gradually decreases to near-ambient conditions (approximately 30-40°C), which reduces its volume by approximately 50% based on the ideal gas law relationship. Meanwhile, the narrower 0.5-meter dimension maintains an appropriate pressure differential (approximately 0.2-0.3 bar) and flow velocity (1.5-2 m/s) that prevents excessive back pressure while ensuring optimal contact with the heat transfer surfaces. The cooled, volume-reduced CO₂ then accumulates in a storage chamber until it is needed for controlled injection into the digestion process. When measured against a more cubic chamber design with equal volume but 1:1 proportion, the 5:1 aspect ratio configuration demonstrates 37% greater thermal efficiency and 42% improved volume reduction capability due to the optimized gas flow dynamics and extended heat transfer contact time. In an embodiment, the modular self-ballasting anaerobic digester apparatus 100 may support the aspect ratio through its standardized connection mechanisms, enabling consistent implementation of the 5:1 proportion across different scales of operation from small 50kg/day units to industrial 30,000kg/day installations.

In an embodiment, the aspect ratio may be achieved by arranging the interceptor tank 101, digester tank 102, and carbon capture apparatus components 105 and 106 in a specific configuration. This optimal aspect ratio may contribute to the apparatus's 100 structural stability and efficient use of space. For example, suppose apparatus 100 is designed to be 5 meters long and 1 meter wide. In that case, interceptor tank 101 may occupy the first 1.5 meters of the length, digester tank 102 may occupy the middle 2 meters of the length, and the carbon capture components (105 and 106) may occupy the remaining 1.5 meters of the length. All components would share the same 1-meter width, maintaining an overall aspect ratio of 5:1 (5 meters long by 1 meter wide). The above-described aspect ratio of 5:1 provides advantages such as efficient use of space, minimal footprint while maintaining operational capacity, ease of transportation and installation, etc. The 5:1 aspect ratio might be advantageous for installations with space constraints in one dimension, such as narrow pathways or corridors, or when trying to maximize the number of units that can be placed side-by-side in a facility. The specific configuration maintains the process flow from the interceptor tank 101 to the digester tank 102 to the carbon capture components 105 and 106 and optimizes the overall dimensional proportions of the apparatus 100. In an embodiment, the modular design of the apparatus 100 may enable scalability and adaptability to various site requirements. The mechanical connections between components may facilitate easy assembly and disassembly, enabling the apparatus to be transported and deployed in different locations.

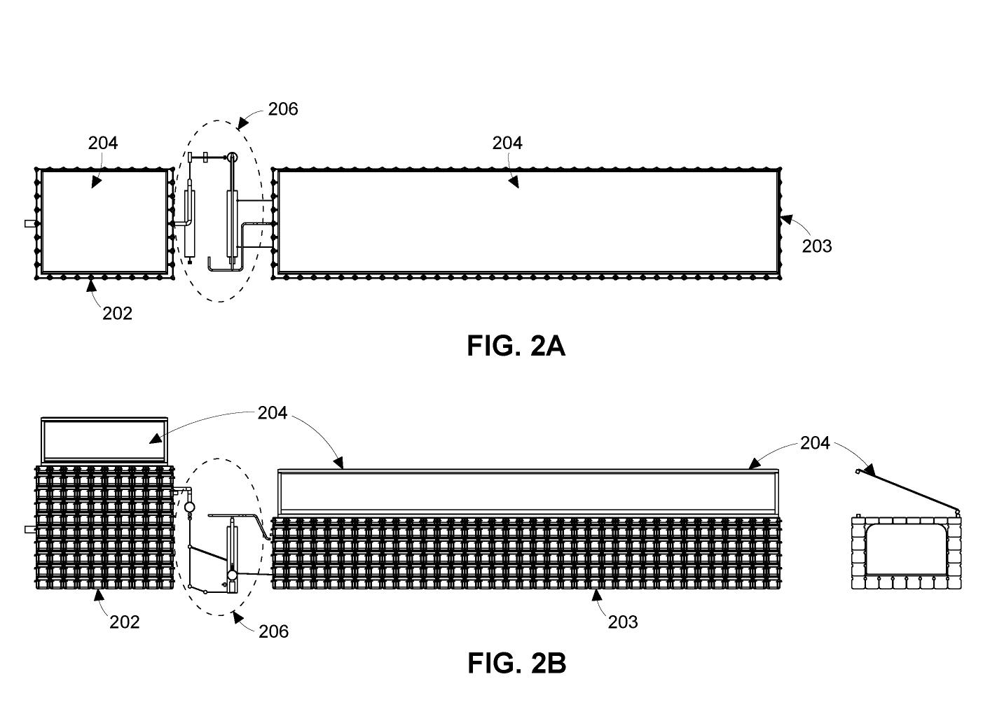

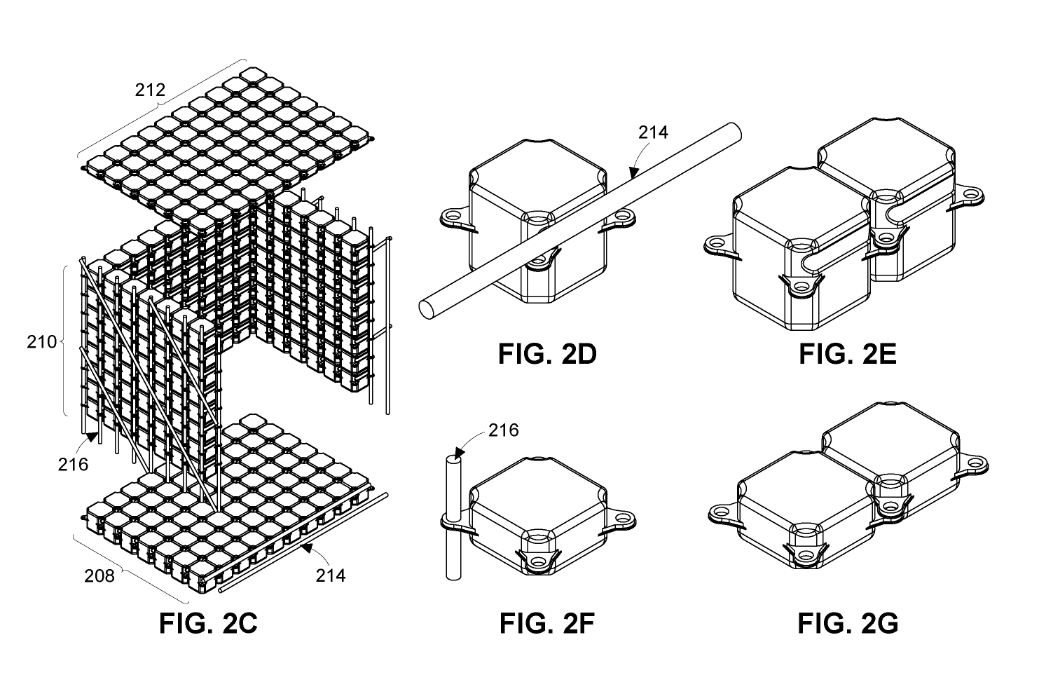

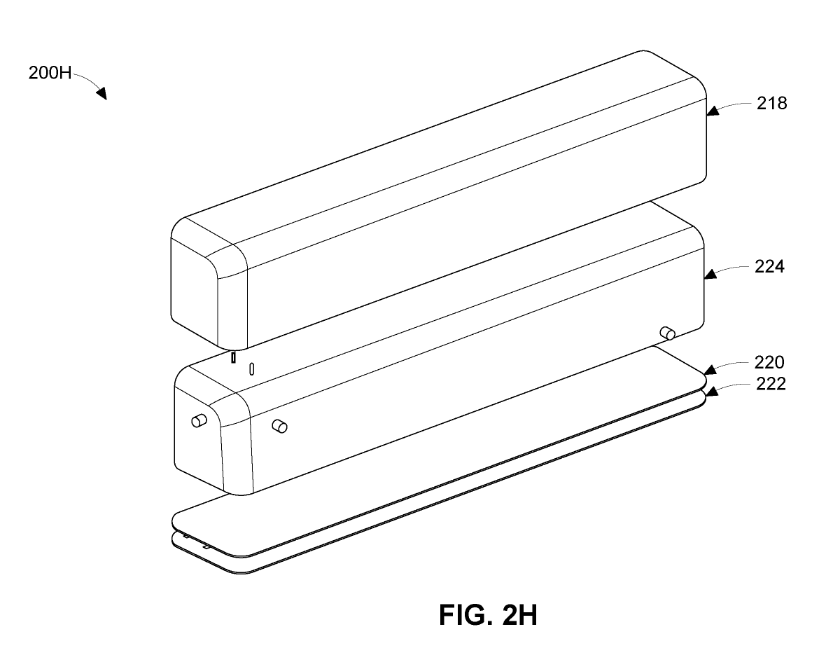

FIGS. 2A and FIG. 2B are illustrations showing orthogonal views of the modular self-ballasting anaerobic digester apparatus according to exemplary embodiments. FIGS. 2A and 2B are described in conjunction with FIGS. 1A and 1B. FIG. 2A shows a top view, and FIG. 2B shows a side view of, for example, an interceptor tank 202 and a digester tank 203. Solar panels 204 are mounted on the roof structure of the apparatus 100. In an embodiment, the digester tank 203 may also be referred to as a composite tanking apparatus 203 or composite tanking system 203. A carbon capture apparatus 206 may be integrated with apparatus 100. The interceptor tank 202 and digester tank 203 may be connected or arranged in a sequential configuration. The solar panels 204 are positioned above the tanks (202 and 203) to capture solar energy. The carbon capture apparatus 206 is located between the interceptor tank 202 and the composite tanks 203, as shown in the dashed circular region.

FIGS. 2C-2G are illustrations showing components of the modular self-ballasting anaerobic digester apparatus, according to an exemplary embodiment. FIG. 2C is an illustration showing an arrangement of gas-filled modules forming the floor module (e.g., 208), wall module (e.g., 210), and roof module (e.g., 212). The structure includes floor module 208, which is mechanically attached and reinforced to create a load-bearing floor structure that is operable to operate or function as a ballast. The wall module 210 is formed by the mechanical attachment of the gas-filled modules, thereby forming the load-bearing walls. Roof module 212 may include low floats that are mechanically attached and reinforced to create a load-bearing roof structure.

In an embodiment, the gas-filled modules may be mechanically connected using torque tubes (e.g., represented by 214 in FIG. 2D and 216 in FIG. 2F). The torque tubes (e.g., 214 and 216) may be operable to provide structural support and also function as guttering for liquid collection. The torque tubes (e.g., 214 and 216) may be made of a durable, corrosion-resistant material such as stainless steel or high-density polyethylene (HDPE). The diameter of the torque tubes may range from, for example, 50 mm to 150 mm, depending on the structural requirements and expected liquid flow rates.

FIG. 2D is an illustration showing a higher-volume gas-filled module, and FIG. 2E is an illustration showing the mechanical connection and reinforcement of the higher-volume gas-filled modules. The higher-volume gas-filled modules may be mechanically connected to facilitate load-bearing and ballast structures. FIG. 2F is an illustration showing a lower-volume gas-filled module, and FIG. 2G is an illustration showing the mechanical interconnection and reinforcement of lower-volume gas-filled modules. The lower-volume gas-filled modules may be mechanically connected to function or operate as load-bearing roof modules (e.g., 212).

For example, the gas-filled modules configuration may include multiple interconnected higher-volume gas-filled modules with a volume-to-weight ratio of approximately 32 floats per ton of supported load. The gas-filled modules may be fabricated with gas-tight seams and configured to maintain positive buoyancy when assembled into the modular framework. The higher-volume configuration is characterized by the gas-filled modules dimensioned with an aspect ratio of 5:1, wherein the gas-filled modules are attached along their upper surfaces, side surfaces, and longitudinal surfaces to form a structure, as shown in FIG. 1A. In an embodiment, the higher-volume gas-filled modules, with capacities of 2.5m³, may be used in larger installations where operational efficiency benefits from fewer connection points and reduced assembly time. A large-scale installation processing 10,000kg of organic waste daily might include 120 higher-volume modules in its structure. The higher-volume gas-filled modules provide enhanced thermal stability due to their greater gas volume, which serves as an insulative barrier against ambient temperature fluctuations. The higher-volume gas-filled modules may provide greater structural rigidity when configured in the apparatus’s standard 5:1 aspect ratio formation, enabling taller installations without compromising stability or requiring additional reinforcement.

For example, the lower-volume gas-filled modules configuration may use a reduced gas-to-structure ratio and maintain the self-ballasting functionality. The lower-volume gas-filled modules configuration may be utilized for smaller-scale implementations processing approximately 50-500 kg of organic matter per day. The lower-volume gas-filled modules, typically measuring 0.5m³ in capacity, may be deployed in installations where space constraints or weight distribution requires more precise ballasting. The lower-volume gas-filled modules may provide enhanced maneuverability during installation. For example, a small-scale digester processing 50 tons of organic waste daily might utilize 24 lower-volume modules arranged in a 3×4×2 configuration to create a stable platform with optimal weight distribution. The reduced individual volume enables manual positioning without mechanical assistance and provides sufficient buoyancy when installed in water-logged environments. The lower-volume gas-filled modules interconnect via the standardized torque tube system, where each connection point can withstand 450kg of vertical load and function or operate as a water collection channel.

In an embodiment, the utilization of the same torque tube connection arrangement may include the torque tubes (e.g., 214 and 216 as shown in FIG. 2D and FIG. 2F) with drainage outlets, such that the torque tubes function simultaneously as structural elements and liquid collection conduits. In an embodiment, the integration with a heat pipe system for thermal regulation and carbon dioxide capture may ensure that the heat transfer efficiency remains consistent with larger configurations.

In an embodiment, the modular self-ballasting anaerobic digester apparatus 100 may utilize different sizes of gas-filled modules to create a versatile and structurally sound container. The higher-volume gas-filled modules, as shown in FIG. 2D and FIG. 2E may be designed to provide the primary structural support and ballast for the apparatus. These higher-volume gas-filled modules may be mechanically connected using multiple fastening means or mechanisms that may include interlocking joints, bolts, or specialized connectors. The mechanical connections ensure that the higher-volume gas-filled modules work together to distribute loads evenly and maintain the overall stability of the structure. In an embodiment, the lower-volume gas-filled modules, as shown in FIG. 2F and FIG. 2G may be designed for use in the roof structure. These lower-volume gas-filled modules may also be mechanically interconnected, but their primary application is to create a lightweight yet strong roof assembly. The roof modules (e.g., 212) may bear loads such as snow, rain, or the weight of solar panels and maintain the gas-tight integrity of the apparatus 100.

In an embodiment, the use of lower-volume gas-filled modules and higher-volume gas-filled modules enables the optimization of the structure's weight distribution and load-bearing capacity. The higher-volume gas-filled modules in the lower portions of the apparatus 100 may provide a stable base and ballast, and the lower-volume gas-filled modules in the roof reduce the overall weight of the upper structure and maintain structural integrity. This design approach enables the modular self-ballasting anaerobic digester apparatus 100 to operate or function as a self-ballasting structure, thereby eliminating the need for additional foundational support or anchoring. In an embodiment, the gas-filled modules may be designed to withstand the forces exerted by the contained gases, the weight of the organic matter being processed, and any external environmental factors, such as wind or seismic activity. Further, the mechanical connections may enable the maintenance of a gas-tight seal to prevent leakage and ensure the efficiency of the anaerobic digestion process. In an embodiment, the mechanically connected gas-filled modules may form the core structural elements of the modular self-ballasting anaerobic digester apparatus 100. The gas-filled modules may be designed to provide both structural integrity and buoyancy, eliminating the need for traditional foundations or extensive site preparation.

FIG. 2H is an illustration showing an exploded view of a composite tanking apparatus, according to an exemplary embodiment. In an embodiment, FIG. 2H is an illustration showing an exploded view 200H of the composite tanking apparatus. In an embodiment, the composite tanking apparatus or the composite tanking system may include the interceptor tank 202 and/or the digester tank 203. As shown in FIG. 2H, the composite tanking apparatus may include an outer blast mitigation layer 218, a gas-absorbent insulation layer 220 (also referred to as an insulation layer), a heat transfer layer 222, and a digestion bladder 224. The digestion bladder 224 may interchangeably also be referred to as a digestion layer in the subject specification.

In an embodiment, the outer blast mitigation layer 218 may be made of a woven fabric with ceramic spheres (not shown) that may be designed to protect against blasts. The gas-absorbent insultation layer 220 may be positioned inside the outer blast mitigation layer and may be operable to absorb gases and provide insulation. The heat transfer layer 222 may include a sealed bladder with heat pipes located within the gas-absorbent insulation layer, operable for transferring thermal energy to the digestion process. The digestion bladder 224 may include a flexible bladder system that stores the organic matter and provides a medium for anerobic digestion. The digestion bladder 224 may also be positioned on top of the heat transfer layer.

In an embodiment, the above-described layers (e.g., 218, 220, 222, and 224) form the internal structures of the apparatus 100 that include mechanically connected gas-filled modules, as explained above. The outer blast mitigation layer 218 may be the outermost part of the apparatus 100, followed by the gas-absorbent insulation layer 220, which is disposed within the outer blast mitigation layer. The heat transfer layer 222 is in the gas-absorbent insulation layer, with a sealed bladder containing heat pipes, indicating a nested structure within the insulation layer. The digestion bladder 224 is positioned on top of the heat transfer layer, positioned above or inside relative to the heat transfer layer, containing the organic matter.

In an embodiment, the composite tanking apparatus, including the interceptor tank 202 and the digester tank 203, may be formed by interconnecting gas-filled modules and include an arrangement of the outer blast mitigation layer 218 forming the external shell (e.g., walls, roof, floor) and providing structural protection. Further, the gas-absorbent insulation layer 220 may line the interior, manage gases, and insulate the contents. Further, within the gas-absorbent insulation layer 220, the heat transfer layer 222 may include a sealed bladder with heat pipes that may be positioned at the bottom or lining the inner surface and operable to regulate temperature. Further, the digestion bladder 224 may include a flexible bladder that may be positioned on top of the heat transfer layer inside the apparatus 100 and operable to hold the organic matter for the anerobic digestion process.

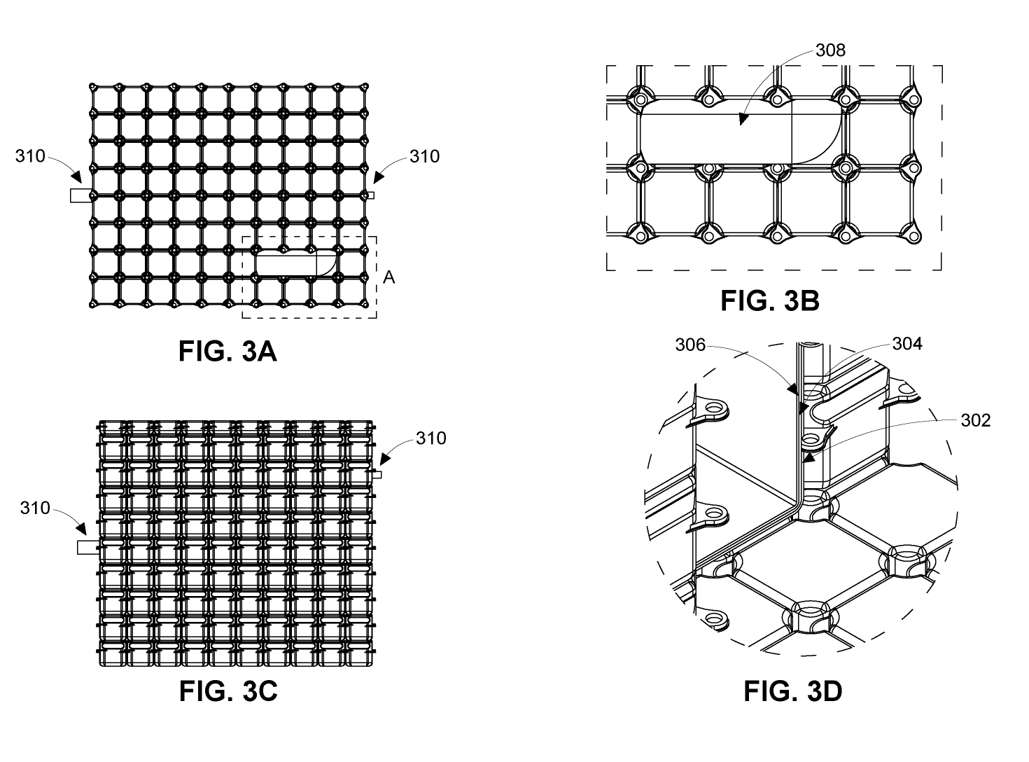

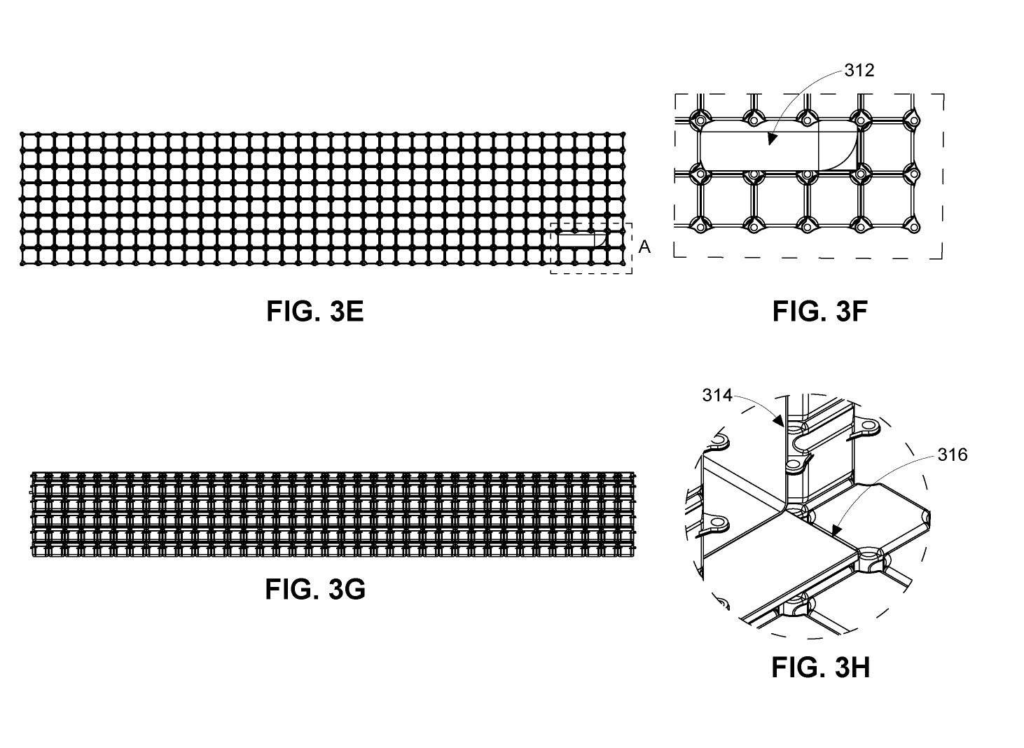

FIGS. 3A and 3B are illustrations showing mechanically connected lower-volume gas-filled modules, according to an embodiment. FIG. 3A shows a top view of the arrangement of the mechanically connected lower-volume gas-filled modules. FIG. 3A shows the interconnection of the lower-volume gas-filled modules to create a stable platform. The blown-up sectional view in FIG. 3B shows a blown-up sectional view illustrating a bladder gap (e.g., 308) between adjacent lower-volume gas-filled modules. The bladder gap (e.g., 308) serves multiple purposes, including enabling thermal expansion and contraction of the lower-volume gas-filled modules, facilitating the circulation of air or other gases between lower-volume gas-filled modules for pressure equalization, and providing channels for routing utilities or monitoring equipment.

FIG. 3C and FIG. 3D are illustrations showing mechanically connected lower-volume gas-filled modules, according to exemplary embodiments. In an embodiment, FIG. 3C shows a side view illustrating insight into the vertical stacking and connection of the lower-volume gas-filled modules. FIG. 3D shows a blown-up sectional view of the internal structure and interconnections. The blown-up or detailed view illustrates the multi-layered composition of the lower-volume gas-filled modules, consisting of an outer layer 302 (also interchangeably referred to as the outer blast mitigation layer), a bind layer 304, and an inner layer 306. In an embodiment, the outer layer 302 may be adapted as the primary barrier against environmental factors and may include materials for UV resistance, weather protection, and potentially the blast mitigation properties mentioned elsewhere in the specification. The bind layer 304 may be adapted to be a structural component that provides rigidity and strength to the module, potentially incorporating reinforcing materials or a honeycomb structure for optimal strength-to-weight ratio. The inner layer 306 may be adapted to contain the gas that provides buoyancy and may be operable to maintain gas pressure and prevent leakage.

In an embodiment, the above-described layers (e.g., 304, 306, and 308) may be operable or work in conjunction to create a robust, lightweight structure capable of supporting the modular self-ballasting anaerobic digester apparatus 100 components and also providing the necessary buoyancy for the self-ballasting feature of the apparatus 100. The mechanical connections between lower-volume gas-filled modules may provide and maintain the structural integrity of the modular self-ballasting anaerobic digester apparatus 100 and may include specialized fasteners or interlocking mechanisms to withstand the forces exerted by the contained gases, organic matter, and environmental factors such as wind or seismic activity.

FIGS. 3E – 3H are illustrations showing interconnected higher-volume gas-filled modules, according to exemplary embodiments. FIG. 3E is an illustration showing a top view of interconnected higher-volume gas-filled modules. FIG. 3F is an illustration showing a blown-up section view of a typical bladder gap (e.g., 312). In an embodiment, the insulation layer 314 is a component of the interconnected higher-volume gas-filled module positioned between the digester bladder (inner layer) and the heater bladder (outer layer). The insulation layer 314 (also interchangeably referred to as a gas-absorbent insulation layer 314) is in the modular self-ballasting anaerobic digester apparatus 100 and may be operable or adapted to provide thermal insulation. The insulation bladder 314 may be operable to maintain optimal temperature conditions within the digester bladder by minimizing heat loss to the surrounding environment. This thermal barrier enhances the overall energy efficiency of the apparatus 100 by reducing the amount of heat input required to maintain ideal digestion temperatures. The insulation layer 314 in the modular self-ballasting anaerobic digester apparatus may be operable or adapted to provide energy conservation. By preventing heat transfer between the digester bladder and the external environment, the insulation bladder contributes to energy conservation within the apparatus 100. This results in lower operational costs and improved sustainability of the anaerobic digestion process. The insulation layer 314 in the modular self-ballasting anaerobic digester apparatus 100 may be operable or adapted to provide temperature stability. The insulation layer 314 may enable buffering against external temperature fluctuations, maintaining a more stable internal environment for the microbial communities responsible for anaerobic digestion. This stability promotes consistent biogas production and overall apparatus performance.

In an embodiment, the insulation layer 314 in the modular self-ballasting anaerobic digester apparatus 100 may be operable or adapted to provide structural support. The insulation bladder also provides additional structural integrity to the interconnected gas-filled module assembly. The insulation layer 314 may facilitate the distribution of loads and pressures more evenly across the module structure. The insulation layer 314 in the modular self-ballasting anaerobic digester apparatus 100 may be operable or adapted to provide leak prevention. For example, in the event of a small leak in either the digester bladder or the heater bladder, the insulation layer 314 may act as a secondary containment barrier, preventing the spread of liquids or gases between layers and potentially mitigating environmental contamination risks. The insulation layer 314 may be typically constructed from materials with low thermal conductivity, such as closed-cell foam or synthetic fibers. The specific material choice and thickness of the insulation layer are optimized to balance thermal performance, weight considerations, and the apparatus's 100 efficiency within the modular self-ballasting anaerobic digester design.

FIGS. 3E-3H illustrate interconnected higher-volume gas-filled modules, showing both top and side views of the interconnected higher-volume gas-filled modules, along with blown-up sectional views illustrating the internal configuration. The higher-volume gas-filled modules may include a bladder gap 312 between adjacent higher-volume gas-filled modules, which provide spacing and enable thermal expansion and contraction. In an embodiment, the internal structure comprises multiple layers, including a digester bladder 314, which is adapted to operate or function as the inner layer where the digestion process occurs. A heater bladder 316 forms the outer layer and contains heating fluid for maintaining temperature through convection.

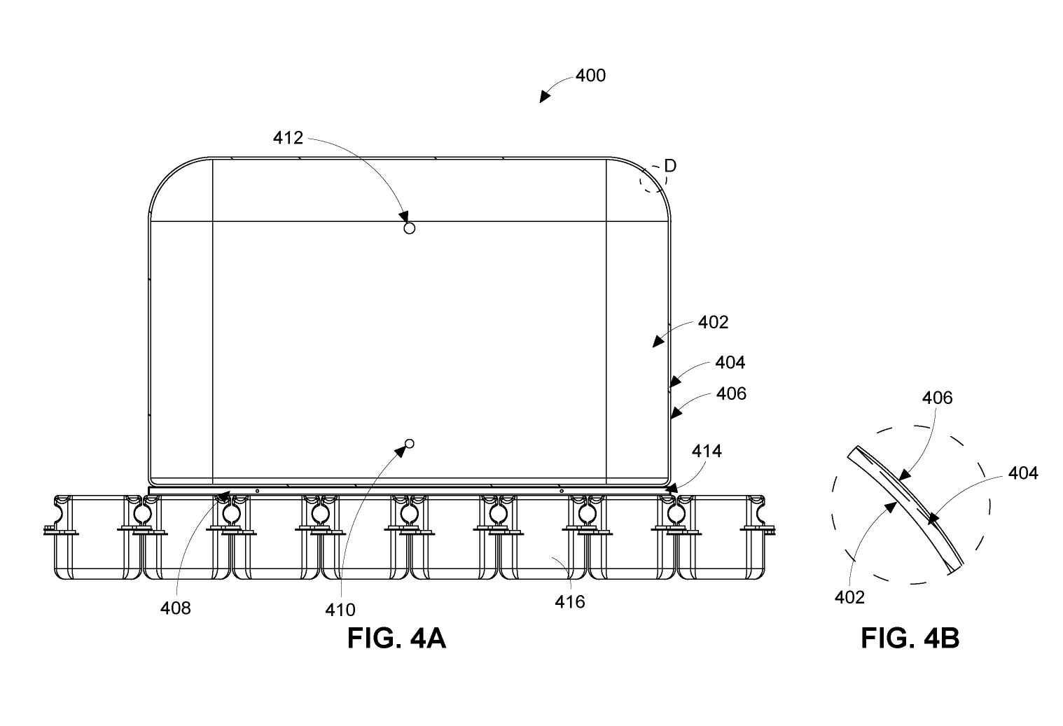

FIG. 4A and FIG. 4B are illustrations showing a sectional view of a gas-filled module 400, according to an exemplary embodiment. FIGS. 4A and 4B show the structure and arrangement of multiple layers and components of gas-filled module 400. In an embodiment, the gas-filled module 400 includes a digester bladder 402 positioned within the structure. An insulation layer 404 surrounds the digester bladder 402, and a woven liner 406 forms the outer layer of the assembly. In an embodiment, the gas-filled module 400 may include a woven fabric layer that acts as an insulator and leak-absorbing panel. This layer may be made of a high-performance synthetic material such as aramid or polyethylene terephthalate (PET) fibers. The fabric may have a thickness ranging from 2 mm to 10 mm and a density of 200 to 600 grams per square meter, providing effective insulation and leak absorption properties.

In an embodiment, the outer layer may include a blast mitigation layer. This layer may be made of a woven liner with ceramic spheres coated with expanding foam. The ceramic spheres may have a diameter ranging from 1 mm to 5 mm and may be made of materials such as alumina or zirconia. The expanding foam coating may be a fire-resistant polyurethane foam with a thickness of 0.5 mm to 2 mm. This blast mitigation layer may protect against potential explosions or impacts, enhancing the overall safety of the apparatus 100.

In an embodiment, the gas-filled module 400 is shown in FIG. 4A may form the basic structural unit of the apparatus 100. As explained above, the gas-filled module 400 may be mechanically connected to create the container structure, including the load-bearing space, roof, walls, and floor. The gas within these gas-filled modules may provide buoyancy, enabling the apparatus 100 to have a self-ballasting feature. The digester bladder 402 may facilitate the anaerobic digestion process. The digester bladder 402 may be heated indirectly by the heating bladder through convection, ensuring optimal temperature conditions for microbial activity. The heat absorbed by the feedstock promotes the efficient breakdown of organic matter and biogas production.

In an embodiment, the middle insulation layer 404 may maintain thermal efficiency. By reducing heat loss or gain, the middle insulation layer 404 may maintain stable temperatures within the digester bladder, which is essential for consistent microbial activity and biogas production. The outer woven liner 404 may provide structural integrity to the module and may act as a protective layer against external forces. The blast mitigation properties of this layer enhance the safety of the apparatus 100, particularly in environments where gas accumulation could pose risks. In an embodiment, heat pipe 408 may be operable to provide the apparatus's 100 efficient thermal management. The heat pipe 408 may be sealed and may transfer heat from external sources or the heating bladder to the digester bladder, ensuring even heat distribution and optimal digestion conditions. In an embodiment, outlet 410 and inlet 412 enable the controlled flow of materials in and out of the gas-filled module. This enables apparatus 100 to manage the input of organic matter and the extraction of digested material and biogas. The heating bladder 414 may include a heating fluid that transfers heat to the digester bladder through convection. The design enables efficient and controllable heating of the digestion process without direct contact between the heating element and the organic matter.

In an embodiment, a float base 416 provides stability and buoyancy to the entire module. The float base 416 may facilitate the provision of the self-ballasting function of apparatus 100, enabling deployment in various environments without the need for extensive foundational work. The modular design, including the layered approach to insulation, heating, and structural integrity, enables the apparatus 100 to be scalable, efficient, and adaptable to various organic waste processing needs. In an embodiment, the heat pipe 408 extends through the apparatus 100 to facilitate heat transfer. In an embodiment, the gas-filled module 400 may include an outlet 410 and an inlet 412 for material flow. A heating bladder 414 is positioned to provide heat to the digester bladder 402. The assembly is supported by the float base 416.

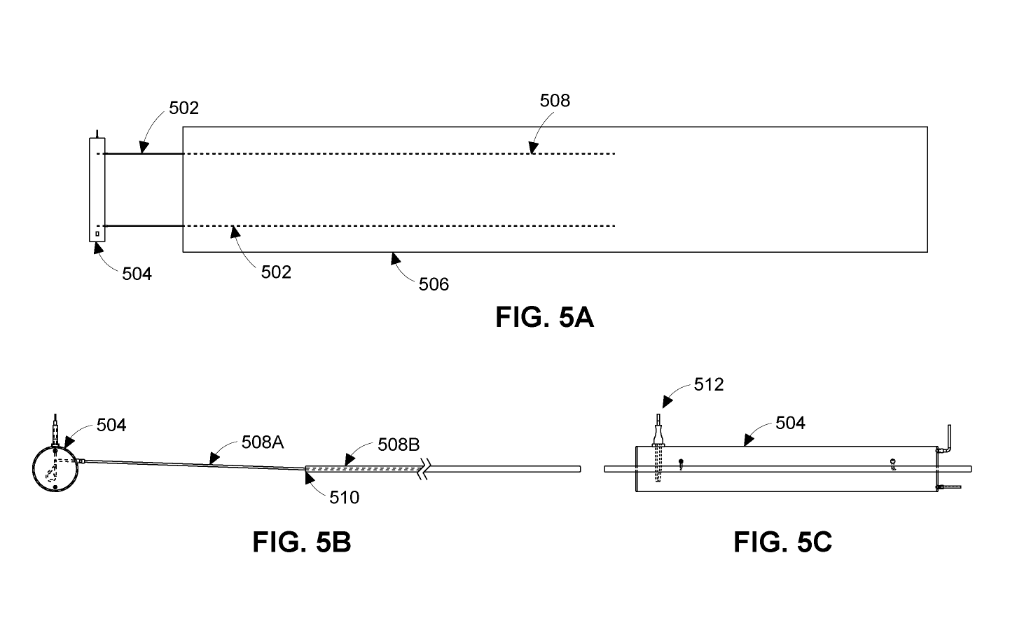

FIG. 5A, FIG. 5B and FIG. 5C are illustrations showing different views of heat transfer components, according to exemplary embodiments. In an embodiment, the heat transfer components may include a heat transfer pipe 504, including a heat transfer medium 502. A heating bladder 506 may be positioned along the heat transfer pipe 504. A heat pipe 508 extends through the apparatus 100 to facilitate heat transfer. The heat pipes (e.g., 508) may use a phase-change medium to transfer heat in a specific direction. The phase-change medium may include a fluid such as water, methanol, or a specialized heat transfer fluid. The heat pipes (e.g., 508) may operate based on the principle of evaporation and condensation, enabling efficient heat transfer with minimal temperature gradient. The heat pipes (e.g., 508) may have a diameter ranging from 10 mm to 50 mm and a length of 0.5 m to 3 m, depending on the specific heat transfer requirements of the apparatus 100.

In an embodiment, the modular self-ballasting anaerobic digester apparatus 100 may capture heat from the solar panels (e.g., 103 in FIG. 1B) and use the guttering for water collection. The solar panels (e.g., 103 in FIG. 1B) may be mounted on the roof structure of the apparatus 100 and may be connected to a network of heat pipes that transfer excess heat to the digestion process. The guttering formed by the torque tubes (e.g., 214 and 216) may collect rainwater and condensation from the solar panels (e.g., 103 in FIG. 1B), directing this water to the apparatus 100.

In an embodiment, the heat pipe 508 in the modular self-ballasting anaerobic digester apparatus 100 may include two main parts, for example, the evaporator 508A and the condenser 508B. The evaporator 508A section of the heat pipe 508 is where heat is absorbed from the source, causing the working fluid inside to vaporize. The condenser 508B section of the heat pipe 508 is where the vapor releases its latent heat and condenses back into a liquid. This two-phase heat transfer mechanism enables efficient and rapid transfer of thermal energy across the apparatus 100. The evaporator 508A and condenser 508B may work together in a closed loop, with the working fluid continuously cycling between liquid and vapor states. This mechanism enables the heat pipe 508 to transfer heat with minimal temperature difference, thereby maintaining precise temperature control in the digestion layer.

In an embodiment, the bladder 510 may be operable to function as a splitter, thereby separating the evaporator 508A and the condenser 508B sections or providing a means to direct the flow of the working fluid. This design may enable greater control over the heat transfer process and enable the apparatus 100 to adapt to varying heating or cooling needs. In addition to the heat pipe 508, the apparatus 100 may also include a heat transfer pipe 504 that can both transfer and absorb/adsorb heat. This versatility enables the apparatus 100 to manage thermal energy efficiently, either adding heat to the digestion process or removing excess heat as needed to maintain optimal conditions for anaerobic digestion. In an embodiment, the immersion heater 512 may provide an additional means of introducing heat directly into the apparatus 100. The immersion heater 512 may be used to supplement the heat provided by the heat pipes or to quickly raise the temperature of the digestion layer when necessary, ensuring that the anaerobic digestion process remains within the desired temperature range for optimal microbial activity and biogas production.

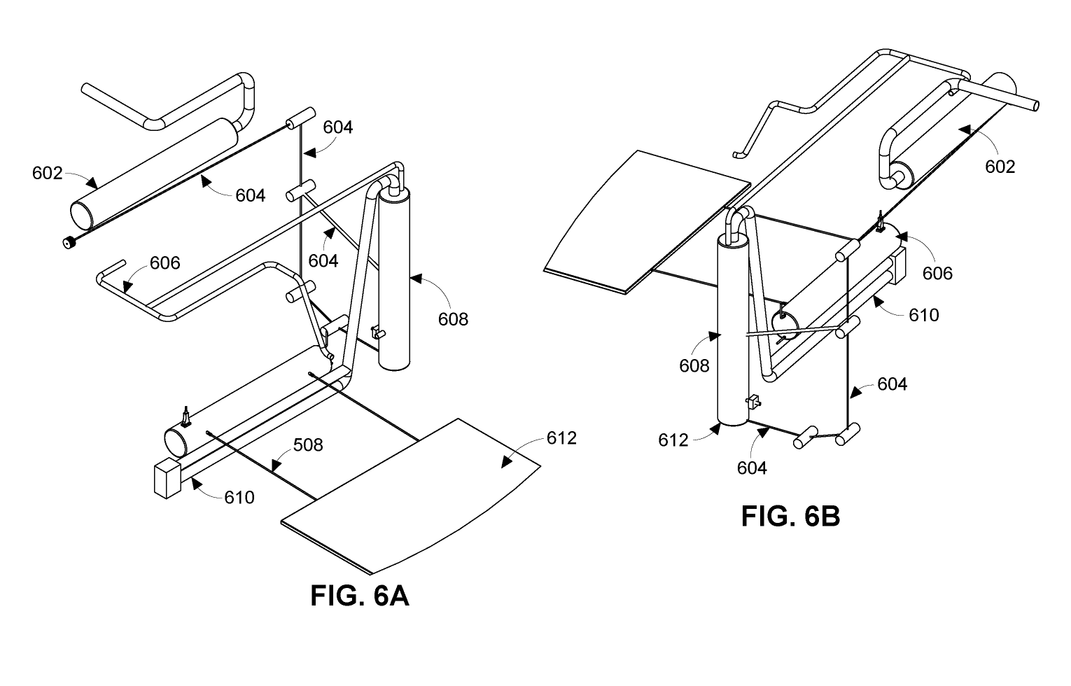

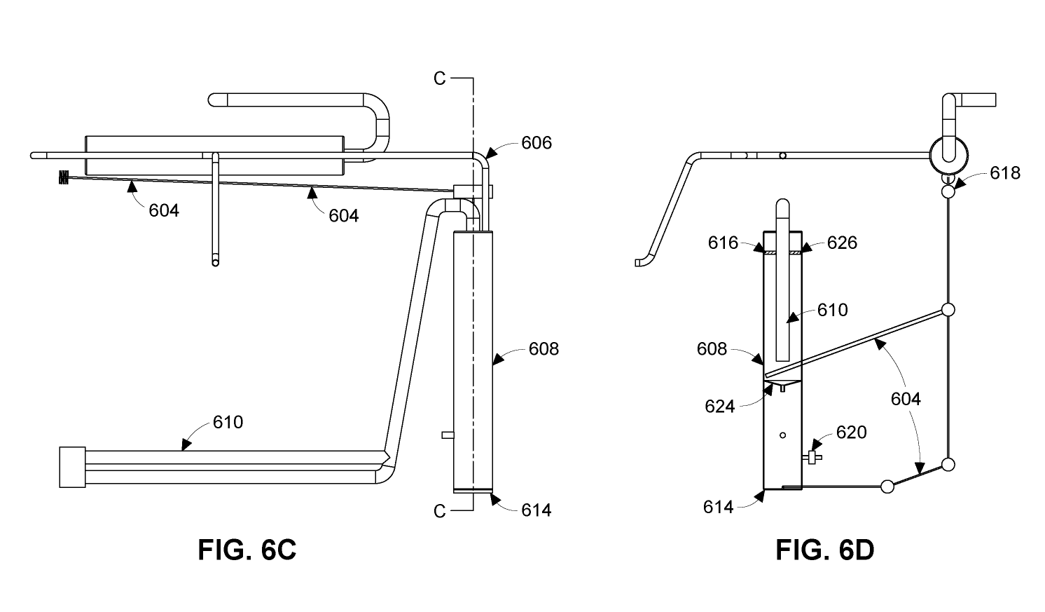

FIGS. 6A and 6B are illustrations showing a carbon capture apparatus that includes multiple interconnected components, according to exemplary embodiments. The carbon capture apparatus may include a pasteurization tank 602 connected to heat pipes 604 (also referred to as carbon capture heat pipes 604). The carbon capture heat pipes 604 facilitate heat transfer within the apparatus 100. A gas injection apparatus 606 may be included to manage the flow of CO2 and other gases. The carbon capture apparatus may include a thermal storage tank 608 that functions to transfer heat and reduce gas volume.

FIG. 6C and FIG. 6D are illustrations showing a sectional view of the carbon capture apparatus, according to exemplary embodiments. In an embodiment, the carbon capture apparatus includes carbon capture heat pipes 604 that facilitate heat transfer within the carbon capture apparatus. A gas injection apparatus 606 is provided to introduce gas into the carbon capture. The thermal storage tank 608 contains components for reducing the velocity and temperature of gases and extracting condensation. The carbon capture apparatus may use the carbon capture heat pipes 604 to cool and reduce the volume of CO₂ gas for storage. As the CO₂ gas passes through the apparatus 100, it may come into contact with the carbon capture heat pipes 604, which may extract thermal energy from the gas. This process may cool the CO₂, reducing the volume and making it easier to store. The cooled CO₂ may then be stored in the thermal storage tank 608 until it is needed for injection into the digestion process.

In an embodiment, the carbon capture apparatus in the modular self-ballasting anaerobic digester apparatus 100 may be designed to capture, cool efficiently, and store CO₂ produced during the anaerobic digestion process, as well as manage heat transfer throughout the apparatus. The pasteurization tank 602 may be used to heat-treat incoming organic material to eliminate pathogens before digestion. This step ensures the safety and efficiency of the anaerobic digestion process. The carbon capture heat pipe's 604 components enable efficient heat transfer throughout the apparatus 100. The carbon capture heat pipes 604 operate on the principle of a thermosyphon, which uses the phase change of a working fluid to transfer heat from one location to another without the need for external pumping. This facilitates balanced load operations by transferring heat from sources of excess thermal energy to areas that require heating. The CO₂ gas injection apparatus 606 may be operable to control precisely the introduction of captured CO₂ or other gases back into the digestion process. This metered injection may enable the optimization of pH levels and enhance biogas production.

In an embodiment, the thermal storage tank 608 may be operable to function as a heat sink to cool the captured CO₂, thereby reducing its volume for more efficient storage. The thermal storage tank 608 may store thermal energy that may be used to maintain optimal temperatures in other parts of the apparatus 100. The thermal storage tank 608 may include components to reduce gas velocity and temperature, as well as extract condensation (e.g., dew). The upper section contains gas, and a lower bypass pipe provides control and safety features. The flue gas feedstock source 610 may be operable to capture heat from exhaust gases. A bypass mechanism ensures that temperatures are maintained at appropriate levels, preventing overheating of the heat pipes at the source. In an embodiment, the condenser 612 may work in conjunction with the carbon capture heat pipes 604 to facilitate the cooling and condensation of gases, particularly CO₂. The flat plate arrangement of the carbon capture heat pipes 604 provides an additional surface area for heat extraction from gases, further reducing their velocity and temperature. A gauze filter 616 may prevent particles from entering the apparatus 100, ensuring only clean gas passes through.

In an embodiment, the thermal store 618 is likely an insulated reservoir that retains captured heat for later use in the digestion process or other heating needs within the apparatus 100. The drain valve 620 enables the removal of accumulated liquids or condensate from the apparatus. The drip tray 624 collects condensation (e.g., dew) that forms during the gas cooling process, thereby preventing it from re-entering the gas stream. The filter 626 removes any remaining solid particles from the gas stream, ensuring that only clean gas is stored or reinjected into the digestion process. The integration of carbon capture and heat management in the apparatus 100 may enhance the overall efficiency of the anaerobic digester by recycling thermal energy and CO2 and also reduce the environmental impact of the digestion process.

In an embodiment, the modular self-ballasting anaerobic digester apparatus 100 may include temperature control mechanisms to maintain optimal microbial activity throughout the digestion process. For instance, the deterministic control system may be programmed to maintain the modular self-ballasting anaerobic digester apparatus 100 within a temperature range of 32°C to 38°C. This temperature range may represent the optimal conditions for the anaerobic bacteria, which may facilitate the bulk of organic matter decomposition in the apparatus 100. For example, when the temperature range of 32°C to 38°C is maintained, the microbial metabolism may remain active but operate at a reduced rate. Further, at the midpoint (35°C), bacteria may achieve optimal metabolic efficiency. Further, at the upper boundary (38°C), the apparatus 100 may approach a transition zone between mesophilic and thermophilic conditions. The carbon capture heat pipes 604 and thermal regulation components ensure uniform temperature distribution throughout the digestion bladder, thereby preventing the formation of cold spots or thermal stratification that could reduce overall efficiency.

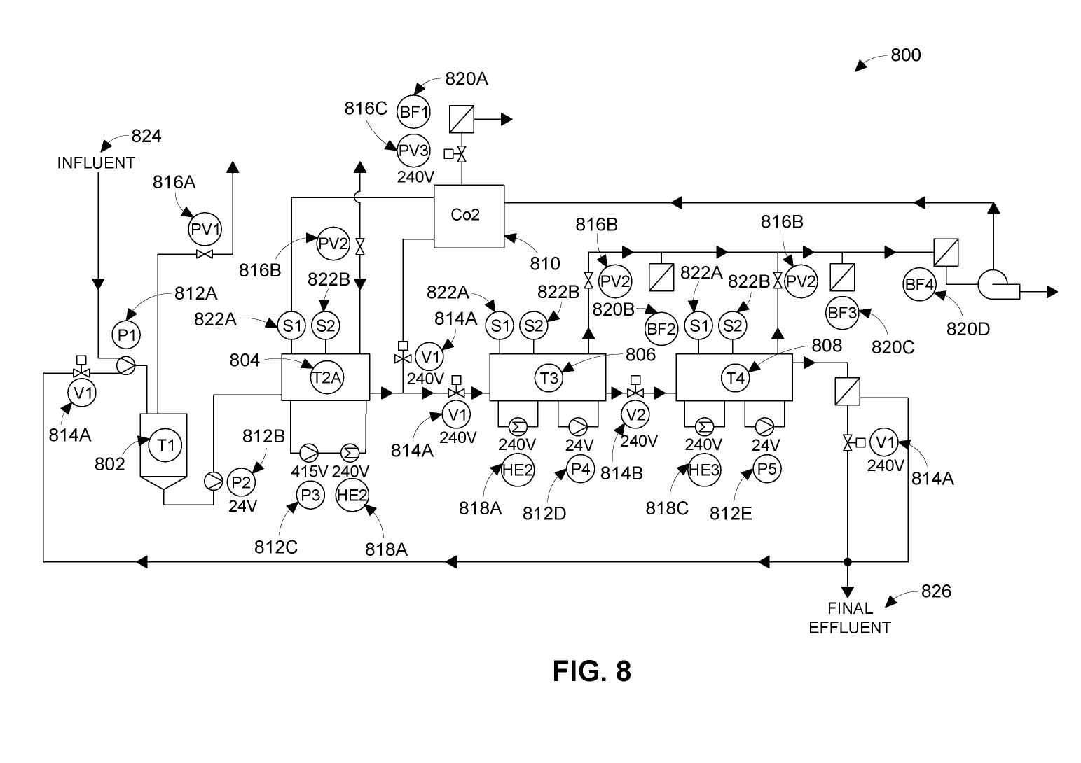

In an embodiment, the modular self-ballasting anaerobic digester apparatus 100 may be configured to operate as a "temperature-phased digester," enabling the apparatus 100 to operate in different temperature ranges and optimize different stages of the digestion process. In an embodiment, the different stages of the digestion process may include a pre-treatment phase, an extended sludge retention phase, a post-digestion processing phase, etc. In the pretreatment phase, the modular self-ballasting anaerobic digester apparatus 100 may implement elevated temperatures in the pre-treatment tank (e.g., tank 1 shown in FIG. 8) to accelerate the hydrolysis of complex organic compounds. In the extended sludge retention phase, the modular self-ballasting anaerobic digester apparatus 100 may be designed to retain solids and enable liquids to pass, creating zones with potentially different temperature profiles to optimize both acidogenesis and methanogenesis. In the post-digestion processing phase, the modular self-ballasting anaerobic digester apparatus 100may implement a thermophilic treatment phase (>55°C) for pasteurization when treating feedstocks that may contain pathogens or when producing fertilizer products that require pathogen reduction.

In an embodiment, the heat pipe throughout the apparatus 100 may transfer thermal energy from multiple sources (e.g., solar panels, waste heat from biogas combustion, ground source, etc.) to the heating bladder positioned beneath the digestion bladder. In an embodiment, the temperature probes may continuously monitor conditions inside the digestion chamber, sending data to the deterministic control system. When the temperature rises above the upper threshold of 38°C, the bypass valve may activate, redirecting thermal transfer until the temperature returns to the acceptable range. The insulation layer surrounding the digestion and heating bladders minimizes heat loss to the environment, reducing energy requirements for maintaining optimal temperature.

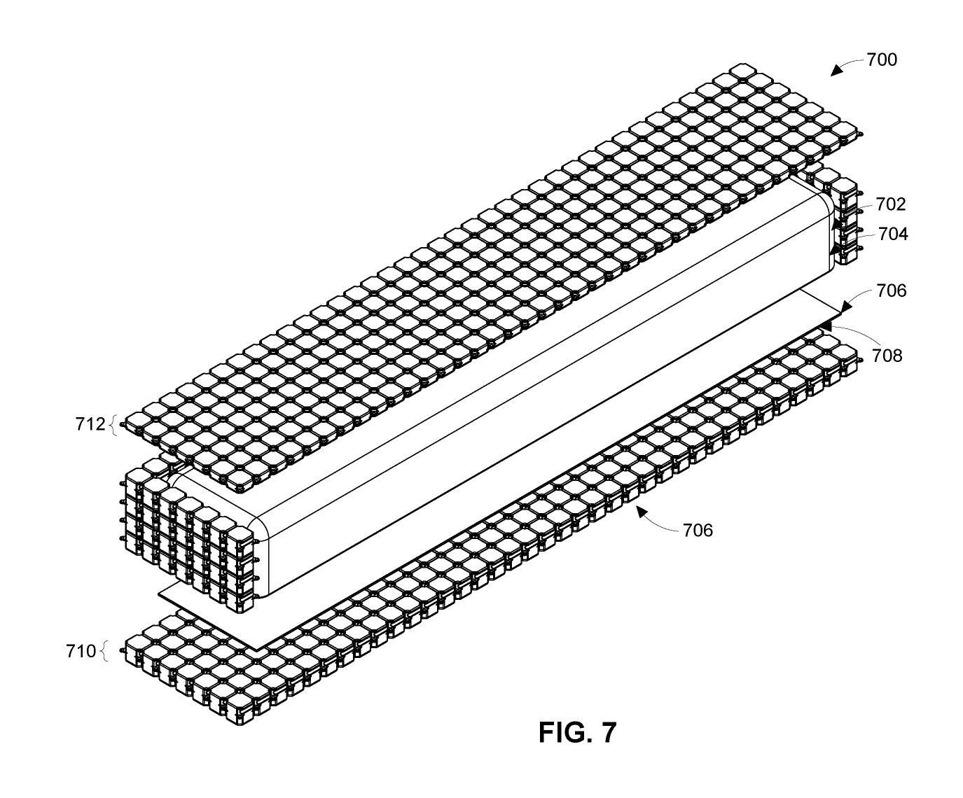

FIG. 7 is an illustration showing an isometric view of the modular self-ballasting anaerobic digester apparatus 100, according to an exemplary embodiment. In an embodiment, the composite tank structure 700 (e.g., the gas-filled modular structure including the interceptor tank 101 and the digester tank 102) may include a bladder 702 and a digester bladder 704 positioned within the structure. A heater layer 706 and a heat transfer layer 708 are arranged to provide temperature control within the modular self-ballasting anaerobic digester apparatus 100. The composite tank structure 700 includes a floor module 710 that provides support for the composite tank 700 components. The floor module 710 may include the load-bearing structure shown in previous figures(e.g., 212 of FIG. 2C). A roof module 712 (e.g., 214 of FIG. 2C) is positioned at the top of the composite tank structure 700, similar to the low floats described in earlier figures(e.g., FIG. 2C). In an embodiment, the arrangement in FIG. 7 shows how the internal space or void enables apparatus 100 to be configured with the various bladder components. The layers may be arranged in a way that enables heat transfer between components and maintains structural integrity through the floor module 710 and roof module 712.

In an embodiment, the deterministic control system may include a programmable logic controller (PLC) operatively coupled to multiple sensors, valves, and pumps. For instance, the deterministic control system may be configured to execute a rule-based algorithm for managing the anaerobic digestion process. The deterministic control system may implement a preventative operation, which may include the process parameters being continuously monitored and adjusted to maintain optimal digestion conditions. For example, the deterministic control system may include a sensor network configured to monitor temperature parameters in the digestion bladder, which may include temperature probes positioned to detect thermal conditions within a range of 32°C to 38°C and level parameters within the composite tanking apparatus. The level sensors may include analog-to-digital converters for precise fluid volume determination. In an embodiment, time parameters relating to retention periods, wherein said time parameters are electronically tracked via the programmable logic controller, and gas composition parameters, such that sensors monitor biogas constituents to determine digestion efficiency and feedstock compatibility.

In an embodiment, the deterministic control system may implement a hierarchical rule structure comprising primary rules relating to temperature maintenance within the digestion bladder. For example, if the temperature deviates from the prescribed range of 32°C to 38°C, the bypass valve is automatically activated until thermal equilibrium is restored. In an embodiment, the secondary rules may be related to level management, such as the predetermined maximum and minimum levels and trigger valve actuation for fluid transfer. The tertiary rules may be related to carbon dioxide injection, which may include a proportional control valve releasing carbon dioxide from the thermal storage tank into the digestion bladder based on, for example, parameters such as current level, composition of the biogas being produced, time elapsed since previous injection, etc.

In an embodiment, the deterministic control system may execute the following operations and may include failure prevention through continuous monitoring of pump and valve operational parameters. For example, when there are deviations from normal operational parameters, an alert may be triggered to the operator, and alternative routing protocols may be initiated. Further execution of operations may include automatic shutdown sequences for individual components when localized component failure does not compromise the integrity of the entire apparatus 100. Further execution of operations may include data lake integration for statistical analysis of operational parameters when the data lake implements pattern recognition algorithms to identify potential failures before they occur. Other operations may include feedstock adaptation protocols, such as when the apparatus 100 automatically adjusts dilution ratios and carbon dioxide injection rates proportional to feedstock characteristics and retention times based on digestion progress indicators.

In an embodiment, the deterministic control system may be physically integrated with the apparatus 100 via, for example, multiple sealed sensor housings penetrating the bladder layers at predetermined measurement points. The deterministic control system may be integrated with the apparatus 100 via sealed wiring conduits channeling sensor data to an environmentally protected control enclosure. Further integrations with the apparatus 100 may include a remote mounting of the programmable logic controller away from the digestion environment to prevent corrosion or damage and modular control architecture, enabling seamless expansion of the apparatus 100. In an embodiment, the deterministic control system may enable the autonomous operation of apparatus 100 with minimal human intervention, which may include the apparatus 100 continuously optimizing the digestion process based on real-time parameter monitoring rather than predefined operational schedules or manual adjustments.

In an embodiment, the modular self-ballasting anaerobic digester apparatus 100 may implement a temperature-phased digestion process with extended sludge retention and metered carbon dioxide injection. This process may optimize biogas production and the breakdown of organic matter. The temperature-phased digestion process may involve multiple stages with different temperature ranges. In an embodiment, the process may begin with a thermophilic phase, where the temperature is maintained between 50°C and 57°C for 2-5 days. This phase may promote the rapid breakdown of complex organic compounds and pathogen reduction. Following the thermophilic phase, the process may transition to a mesophilic phase, where the temperature is lowered and maintained between 35°C and 42°C for 15-25 days. This extended mesophilic phase may enable more stable digestion and improved methane production.

In an embodiment, the modular self-ballasting anaerobic digester apparatus 100 may utilize heat pipes and thermal storage to control and maintain these temperature ranges precisely. In an embodiment, the heat pipes may be filled with a working fluid such as water or a specialized heat transfer fluid with a boiling point between 30°C and 60°C. The heat pipes may have a diameter of 20-40 mm and a length of 1-2 meters, depending on the specific tank dimensions. Extended sludge retention may be achieved through a combination of tank design and process control. The digester tank may include baffles or internal dividers to create zones of different flow velocities. These baffles may be made of corrosion-resistant materials, such as stainless steel or high-density polyethylene, with a thickness of 5-10 mm. The baffles may be arranged to create a serpentine flow path, increasing the effective retention time of solids.

In an embodiment, the modular self-ballasting anaerobic digester apparatus 100 may include a solids recirculation loop. A portion of the digested sludge may be pumped from the outlet of the digester back to the inlet at a rate of 5-15% of the total flow. This recirculation may enable the maintenance of a stable microbial population and improve overall digestion efficiency. Metered carbon dioxide injection may be implemented to enhance biogas production and maintain optimal pH levels within the digester. In an embodiment, a carbon dioxide injection apparatus may include a storage tank, a pressure regulator, and a flow meter. The storage tank may have a capacity of 100-500 liters and be rated for pressures up to 15 bars. The carbon dioxide injection rate may be controlled based on multiple parameters, including pH, alkalinity, and biogas composition. In an embodiment, the injection rate may range from 0.1 to 1.0 liters of CO2 per cubic meter of digester volume per day. The injection point may be located near the bottom of the digester to maximize the dissolution and distribution of the CO₂.

In an embodiment, a pH sensor with an accuracy of ±0.1 pH units may continuously monitor the digester contents. When the pH rises above a set point (typically 7.6-7.8), the deterministic control system may trigger CO₂ injection to lower the pH and maintain optimal conditions for methanogenic bacteria. The biogas produced by the digestion process may be continuously monitored for composition. A gas analyzer capable of measuring methane (CH₄), carbon dioxide (CO₂), and hydrogen sulfide (H₂S) concentrations may be used. The analyzer may have a measurement range of 0-100% for CH₄ and CO₂ and 0-1000 ppm for H₂S, with an accuracy of ±1% of full scale.

In an embodiment, the modular self-ballasting anaerobic digester apparatus 100 may include a pre-treatment mechanism to prepare the organic matter before it enters the main digestion process. This pre-treatment mechanism may include mechanical grinding to reduce the particle size to less than 12 mm, followed by a heat treatment step at 70°C for 1 hour to ensure pathogen reduction. The modular design of apparatus 100 enables easy scaling and adaptation to different waste streams and production volumes. In an embodiment, multiple digester apparatuses 100 may be connected in series or parallel to achieve the desired treatment capacity. Each apparatus 100 may have a volume of 10-50 cubic meters, with the specific size chosen based on the expected waste input and desired retention time.

In an embodiment, the modular self-ballasting anaerobic digester apparatus 100 may be operable through a series of interconnected processes and component interactions to efficiently break down organic matter and produce biogas. The apparatus 100 may utilize heat transfer mechanisms and carbon capture technologies to optimize performance and reduce environmental impact. In an embodiment, the anaerobic digester process may begin in the pre-treatment mechanism, where the organic matter may be mechanically processed to reduce particle size and increase surface area for microbial action. The pre-treatment mechanism may include a combination of grinding and screening mechanisms to achieve a particle size distribution ranging from 1 mm to 12 mm. This size reduction may enhance the efficiency of subsequent digestion stages.

Following pre-treatment, the organic matter may be transferred to the digestion layer within the composite tanking apparatus 100. The digestion layer may maintain specific environmental conditions to promote microbial activity. In an embodiment, the temperature within the digestion layer may be controlled within a range of 35°C to 55°C, depending on the specific microbial populations and desired reaction rates. The pH of the digestion environment may be maintained between 6.8 and 7.5 to support optimal microbial growth and metabolic activity. The heat transfer mechanism may maintain the desired temperature within the digestion layer. Heat pipes sealed within the heat transfer layer may facilitate efficient thermal energy transfer between external heat sources and the digestion environment. The heat pipes may contain a working fluid with a boiling point between 30°C and 60°C, enabling effective heat transfer across the operating temperature range of the apparatus.

In an embodiment, the heat pipes may be arranged in a network configuration to enable uniform heat distribution throughout the digestion layer. The spacing between individual heat pipes may range from 10 cm to 50 cm, depending on the specific thermal requirements and geometry of the digestion vessel. This arrangement may maintain temperature uniformity within ±2°C across the entire digestion volume. The carbon capture apparatus may interact with the digestion process to manage gas production and composition. As the anaerobic digester progresses, biogas containing methane (CH4) and carbon dioxide (CO2) may be produced. The carbon capture apparatus may selectively remove CO2 from the biogas stream, potentially increasing the methane concentration and overall energy value of the produced gas.

In an embodiment, the carbon capture apparatus may include a series of heat pipes and thermal storage tanks to cool and condense the CO₂ component of the biogas. The cooling process may reduce the temperature of the gas stream from the digestion temperature (35°C to 55°C) down to a range of 5°C to 15°C. This temperature reduction may facilitate the separation of CO₂ from the methane-rich biogas. The cooled and captured CO₂ may be stored in a thermal storage tank for later use. The storage tank may maintain the CO₂ at a pressure between 5 bars and 15 bars, enabling efficient storage and controlled release. The stored CO₂ may be metered back into the digestion process to enable the regulation of the pH levels and enhance methane production.

In an embodiment, the deterministic control system may continuously monitor and adjust various parameters to optimize the anaerobic digester process. The parameters monitored may include temperature, pH, gas composition, and liquid levels within the digestion layer. The control system may use these inputs to make real-time adjustments to heating rates, CO2 injection, and organic matter feed rates. For example, if the pH in the digestion layer begins to rise above 7.5, the control system may trigger the release of stored CO2 from the carbon capture apparatus. The CO2 injection rate may be modulated between 0.1 and 1.0 liters of CO2 per cubic meter of digester volume per hour, depending on the magnitude of the pH deviation and the rate of change. The interaction between the heat transfer mechanism and the carbon capture apparatus may create synergistic effects that enhance the apparatus100’s performance. The heat extracted during the CO₂ cooling process may be redirected to the heat transfer layer, potentially reducing the external energy input required to maintain optimal digestion temperatures. This heat recovery process may improve the overall energy efficiency of the apparatus.

In an embodiment, the apparatus 100 may include a biogas recirculation loop to enhance mixing and improve contact between the microbial populations and the organic substrate. The recirculation rate may be adjusted between 10% and 30% of the total biogas production rate, depending on the viscosity of the digestion medium and the desired level of mixing. The modular nature of the apparatus 100 may enable flexible scaling and adaptation to varying input volumes and compositions. Multiple digestion apparatuses 100 may be connected in series or parallel configurations to accommodate different treatment capacities. In a series configuration, each subsequent apparatus 100 may operate at a progressively lower temperature, creating a temperature-phased anaerobic digester process that may improve overall organic matter conversion efficiency.

In an embodiment, the apparatus 100 may be designed to handle a wide range of organic loading rates, from 1 kg to 10 kg of volatile solids per cubic meter of digester volume per day. The specific loading rate may be adjusted based on the composition of the organic input matter and the desired biogas production rate. By carefully controlling and optimizing these various interactions and processes, the modular self-ballasting anaerobic digester apparatus may achieve efficient organic waste treatment, biogas production, and carbon capture capabilities in a compact and adaptable way.

FIG. 8 illustrates a process flow 800 diagram of the modular self-ballasting anaerobic digester apparatus 100, according to an exemplary embodiment. The process flow 800 implementations of the modular self-ballasting anaerobic digester apparatus 100 may include multiple tanks arranged sequentially, for example, including a primary tank 802, a secondary tank 804, a tertiary tank 806, and a quaternary tank 808. A carbon dioxide unit 810 is integrated into the apparatus 100 for gas management. The process flow 800 shows multiple pumps, for example, a first pump 812A, a second pump 812B, a third pump 812C, a fourth pump 812D, and a fifth pump 812E. Control valves 814A and 814B are positioned at various points to regulate flow through the process flow 800.

In an embodiment, the process flow 800 shows multiple valves, for example, a first metering valve 816A, a second metering valve 816B, and a third metering valve 816C for flow control. Further, the process flow 800 diagram shows multiple heating elements, for example, a first heating element 818A, a second heating element 818B, and a third heating element 818C are distributed throughout the apparatus 100 to maintain temperature control. The process flow diagram shows multiple blowers, for example, a first blower 820A, a second blower 820B, a third blower 820C, and a fourth blower 820D may be included in the apparatus for gas movement. The process flow 800 diagram shows monitoring capabilities through, for example, a first sensor 822A, and a second sensor 822B, positioned as shown in FIG. 8.

In an embodiment, the material flow shown through the process flow 800 begins at an influent port 824 and proceeds through the various tanks (e.g., 802, 804, 806, 808) and processing stages before exiting through an effluent port 826. The arrangement of components creates a flow path that enables the processing of material from input to output and maintains controlled conditions through the various stages. In an embodiment, the deterministic control system may use deterministic rules based on time, temperature, and level sensors to manage the anaerobic digester process. For instance, the first sensor 822A, and the second sensor 822B, may monitor parameters such as temperature and liquid levels within the tanks. The deterministic control system may use these inputs to adjust the operation of the heating elements (818A, 818B, and 818C), pumps (812A, 812B, 812C, and 812D), and valves (814A, 814B) to maintain optimal conditions for digestion. For example, if the temperature in the primary tank 802 falls below a predetermined threshold, the deterministic control system may activate the first heating element 818A, to raise the temperature. Similarly, suppose the liquid level in the secondary tank 804 exceeds a certain threshold value. In that case, the deterministic control system may open the control valve 814A to enable excess liquid to flow to the next stage.

In an embodiment, the process flow 800 may be designed to handle varying feedstocks by diluting or thickening the material in different tanks. The primary tank 802 may be used for initial feedstock preparation. If the incoming material is too thick, the deterministic control system may activate the first pump 812A to add liquid from a dilution source. Conversely, if the feedstock is too thin, the process flow 800 may extend the retention time of the feedstock in the primary tank 802 to enable natural thickening through evaporation or may introduce thickening agents. The secondary tank 804 and quaternary tank 808 may be operable to provide additional adjustments to the consistency of the digesting material. The deterministic control system may use the metering valves 816A, 816B, and 816C to control the flow rates between tanks (802, 804, 806, and 808), enabling optimal retention times based on the specific characteristics of the feedstock.

In an embodiment, carbon dioxide unit 810 may be operable for managing the pH levels within the apparatus 100. In an embodiment, if the pH in one of the tanks (802, 804, 806, and 808) becomes too alkaline, the deterministic control system may direct the carbon dioxide unit 810 to inject CO₂ into the tank to balance the pH. The proportional valves may control this injection to ensure precise dosing. The blowers 820A, 820B, 820C, and 820D may be used to circulate gases within the apparatus 100, promoting even distribution of heat and facilitating the movement of biomass. Accordingly, the pumps move the biomass which is the source of biogas around the tank to spread the heat and to prevent scum build up. The deterministic control system may adjust the operation of the blowers 820A, 820B, 820C, and 820D based on gas composition readings from the sensors 822A, 822B or pressure differentials between different parts of the apparatus 100.

In an embodiment, the deterministic control system may implement predictive algorithms based on historical data to anticipate changes in feedstock composition or digestion rates. This may enable the apparatus 100 to proactively adjust operating parameters to maintain stable performance even when faced with varying input conditions. The modular nature of the process flow 800 may enable easy scaling and adaptation to different treatment capacities. Additional tanks or processing units may be added to the apparatus 100 and integrated into the control scheme without requiring a complete redesign of the process flow.

In an embodiment, the modular self-ballasting anaerobic digester apparatus 100 may implement a temperature-phased anaerobic digestion process with Extended Sludge Detention (ESD) operating on a sequential batch basis to optimize biogas production and process efficiency. The composite tank arrangement, as shown and described in FIG. 8, enables each tank to maintain specific temperature ranges that are conducive to distinct microbiological processes. The processing tanks (for example, tank 3 and tank 4) operate within a mesophilic temperature range of 32-37°C, which provides optimal conditions for the methanogenic bacteria responsible for biogas production. Tank 2, for example, may operate at a significantly elevated temperature range of 70-75°C, creating thermophilic conditions that facilitate rapid hydrolysis of complex organic compounds, pathogen reduction, and enhanced solubilization of particulate matter prior to introduction into the mesophilic digestion environment.

In an embodiment, the deterministic control system may implement an algorithmic approach based on three primary parameters, for example, time, level, and temperature. These parameters function as decision criteria that determine the timing of tank filling and emptying operations, as well as when maintenance attention or corrective action is required. The programmable logic controller may execute rules-based decisions, drawing upon real-time sensor data. For example, when tank 2 reaches its predetermined maximum level and has maintained a temperature of 70-75°C for a specified period (typically 1-2 hours for pathogen reduction), the control apparatus automatically initiates transfer of the pretreated substrate to tank 3, but only if tank 3's level parameters indicate sufficient capacity, and its temperature remains within the mesophilic range of 32-37°C. This prevents thermal shock to the methanogenic bacteria population, which could otherwise compromise process stability.

In an embodiment, the temperature-phased approach enhances the apparatus100’s performance by subjecting organic waste to an initial high-temperature treatment in tank 2, which accelerates hydrolysis of complex compounds into simpler molecules more readily metabolized by methanogenic bacteria. For instance, when processing food waste containing significant amounts of cellulose and hemicellulose, the thermophilic pretreatment at 70-75°C increases solubilization rates by approximately 40-60% compared to direct mesophilic digestion, resulting in improved biogas yields and reduced retention time requirements. Following this pretreatment, the substrate is introduced to, for example, tank 3, where mesophilic conditions (32-37°C) provide optimal growth conditions for methanogens that convert volatile fatty acids to methane and carbon dioxide.

For instance, the ESD mechanism may operate or function such that the liquid effluent is separated from the active microbial biomass, allowing the latter to remain within the apparatus 100. This is accomplished through the placement of baffles that function as weirs, forcing solids to settle and permitting clarified liquid to flow through. By extending the sludge retention time beyond the hydraulic retention time, the apparatus 100 maintains a consistently high concentration of active microbial biomass, thereby increasing treatment capacity per unit volume. For example, the sludge retention time can be extended to 45-60 days, resulting in a more stable and robust microbial community capable of withstanding variations in organic loading rate and feedstock composition.

In an embodiment, the sequential batch operation may further enhance process stability by enabling control over feeding and extraction cycles. Rather than continuous flow, which can introduce hydraulic and organic loading variations, the batch approach ensures that each volume of substrate undergoes a complete and consistent treatment cycle. The deterministic control apparatus monitors time elapsed since batch initiation, level changes indicating gas production rates, and temperature stability to determine optimal timing for batch completion. When processing highly degradable substrates such as food waste, a typical batch cycle might comprise 24 hours of high-temperature pretreatment in, for example, tank 2 (e.g., as shown in FIG. 8) followed by 15-20 days in the mesophilic environment of, for example tanks 3 and 4 (e.g., as shown in FIG. 8), with the deterministic control apparatus continuously adjusting retention times based on process performance indicators such as biogas composition and production rate.

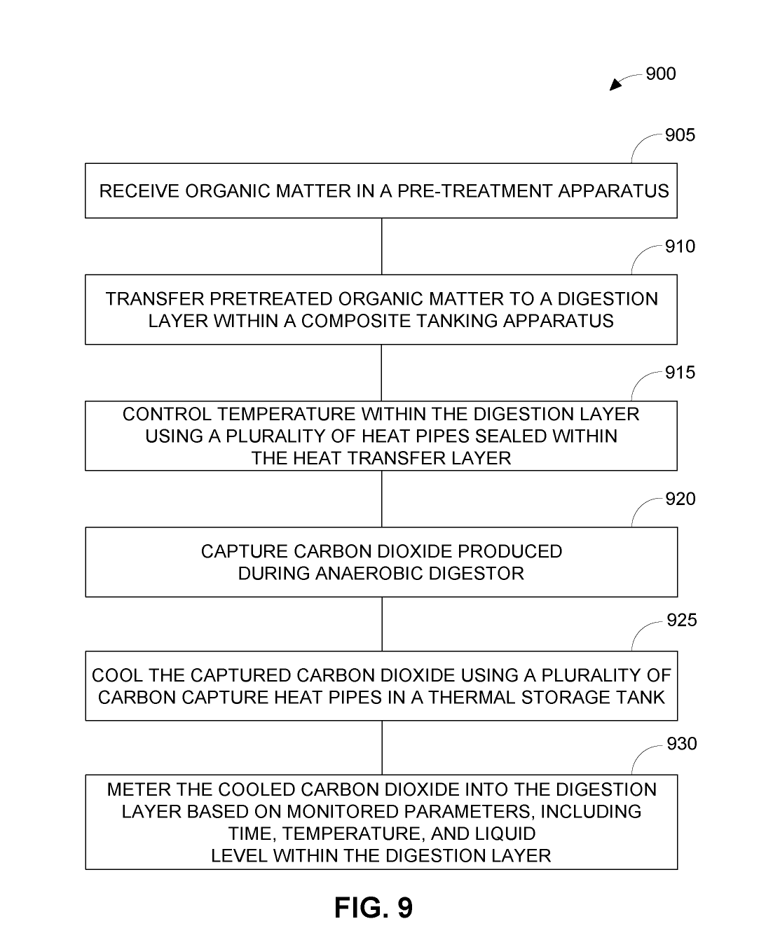

FIG. 9 is a flow chart illustrating a process 900 for operating a modular self-ballasting anaerobic digestor apparatus, according to an exemplary embodiment. FIG. 9 is described in conjunction with FIGS. 1A through FIG. 8. In an embodiment, at step 905, organic matter may be received in a pre-treatment apparatus. This step represents the initial phase where the apparatus 100 accepts various organic waste materials that may undergo processing prior to the main digestion phase. The pre-treatment apparatus may separate, reduce size, or homogenize the organic matter to optimize subsequent digestion. This step may be implemented using microcontroller-controlled mechanical components, such as a programmable logic controller (PLC) that coordinates input sensors, conveyor mechanisms, and processing elements to regulate the intake and preparation of organic material.

At step 910, pretreated organic matter may be transferred to a digestion layer in a composite tanking apparatus of the apparatus 100. This transfer operation moves the processed organic material from the pre-treatment apparatus into the specialized digestion environment, which may be composed of multiple layers, including woven fabric, gas-absorbent material, and bladder apparatus, as described previously. The transfer mechanism may be implemented using a dedicated microprocessor-controlled pumping apparatus with pressure sensors that monitor and adjust flow rates based on the digestion layer's current capacity and processing status.

At step 915, the temperature within the digestion layer may be controlled using a plurality of heat pipes sealed within the heat transfer layer. These heat pipes, functioning as thermosyphons, may transfer thermal energy efficiently to maintain the optimal mesophilic temperature range of 32-38°C required for anaerobic digestion. The heat pipes may operate passively based on their internal phase-change medium. Temperature control may be implemented using a dedicated thermal management mechanism that processes data from temperature probes positioned throughout the digestion layer, controlling bypass apparatus and heat pipe operation to maintain the desired temperature profile across the entire digestion volume.

At step 920, carbon dioxide produced during anaerobic digestion may be captured. This step involves the collection of CO₂ gas generated as a byproduct of the microbial decomposition process occurring within the digestion layer. The carbon capture mechanism may direct the gas through manifolds into a storage apparatus. This capture process may be implemented using an integrated framework of gas composition sensors that analyze the output gas stream in real time, triggering appropriate valves and directing the carbon dioxide to the thermal storage tank and route other gases (for example, methane) to the collection apparatus for energy use.

At step 925, the captured carbon dioxide may be cooled using multiple carbon capture heat pipes in a thermal storage tank. This cooling process reduces the gas volume and prepares it for efficient storage. The cooling mechanism utilizes heat pipes that extract thermal energy from the hot CO₂ gas, transferring it to a thermal storage, which reduces both the temperature and volume of the gas by approximately 50%. This cooling operation may be implemented using a thermal management unit with integrated sensors that monitor heat flow rates, gas temperatures, and condensation levels, dynamically adjusting the thermal pathways to optimize cooling efficiency.

At step 930, the cooled carbon dioxide may be metered into the digestion layer based on monitored parameters, including time, temperature, and liquid level within the digestion layer. This controlled reintroduction of CO₂ enhances the digestion process and biogas production. The metering apparatus regulates the amount and timing of CO₂ injection into the digestion layer for optimal performance. This metering operation integrates data from multiple sensor systems (for example, time controllers, temperature probes, and liquid level sensors) to precisely control the opening and closing of injection valves, determining the optimal timing and quantity of CO₂ to introduce to the digestion process based on the current state of the apparatus.

In an embodiment, the modular self-ballasting anaerobic digester apparatus 100 may be used across various industrial sectors, demonstrating versatility and adaptability to different operational requirements. The apparatus's 100 modular design enables scalability, enabling deployment in a wide range of applications from small-scale operations to large industrial facilities. For example, apparatus 100 may be utilized in the food and beverage industry to process organic waste from production facilities. In an embodiment, a brewery may implement the modular self-ballasting anaerobic digester apparatus 100 to treat spent grain and wastewater, thereby converting these byproducts into biogas for energy recovery. The apparatus's 100 capacity may be adjusted to handle daily organic waste volumes ranging from 50 kg for microbreweries to several thousand kilograms for large-scale brewing operations.

Another application may include, for example, the agricultural sector, which may benefit from the modular self-ballasting anaerobic digester apparatus 100 for managing animal manure and crop residues. In an embodiment, a dairy farm may use the apparatus to process manure from a herd of 100 to 1000 cows, with the apparatus 100 scaled accordingly to handle the daily waste production. The resulting biogas may be used for on-farm energy generation, and the digestate may serve as a nutrient-rich fertilizer for crop production.

Another application may include, for example, municipal waste management facilities that may include the modular self-ballasting anaerobic digester apparatus 100 for processing an organic fraction of municipal solid waste (OFMSW). The modular nature of the apparatus 100 may enable gradual capacity expansion as waste volumes increase over time. In an embodiment, a municipal facility may start with an apparatus processing 1000 kg per day and expand to handle up to 300,000 kg per day as the community grows or as more waste streams are diverted to the anaerobic digester.

Another application may include, for example, apparatus 100, which may find applications in the hospitality industry, where hotels and resorts generate significant quantities of food waste. A mid-sized hotel may implement an apparatus capable of processing 500 kg to 2000 kg of organic waste per day, reducing waste disposal costs and generating renewable energy for on-site use.

Another application may include, for example, the modular self-ballasting anaerobic digester apparatus 100 in the industrial wastewater treatment sector, which may be applied to high-strength organic wastewater streams. For instance, a pulp and paper mill may utilize the apparatus to treat wastewater with high concentrations of organic matter. The apparatus may be scaled to process wastewater volumes ranging from 100 cubic meters to 10,000 cubic meters per day, depending on the size of the industrial operation.

For example, the food processing industry may benefit from the modular self-ballasting anaerobic digester apparatus for managing organic waste from various production lines. A vegetable processing plant may implement the apparatus to handle daily waste volumes from 1000 kg to 50,000 kg, converting vegetable trimmings and rejected produce into biogas and nutrient-rich digestate. In an embodiment, the apparatus may be deployed in remote or off-grid locations where waste management infrastructure is limited. For example, a mining camp in a remote area may use a small-scale version of the apparatus processing 50 kg to 200 kg of organic waste per day, providing a sustainable solution for waste management and energy generation.

In an embodiment, the scalability of the modular self-ballasting anaerobic digester apparatus 100 may enable its application in centralized waste treatment facilities serving multiple industries or communities. A regional biogas plant may utilize multiple modular units to achieve a processing capacity of up to 300,000 kg per day, accepting organic waste from various sources such as agriculture, food processing, and municipal collections.

For example, in the pharmaceutical industry, the apparatus may be utilized to treat organic waste streams from drug manufacturing processes. The modular design may enable pharmaceutical companies to scale the apparatus from pilot-scale operations processing 100 kg per day to full-scale industrial applications handling up to 10,000 kg of organic waste daily. The flexibility in scaling from 50 kg to 300,000 kg per day processing capacity may enable the modular self-ballasting anaerobic digester apparatus to meet the needs of diverse industries and operational scales. This scalability may be achieved through the addition or removal of modular components, enabling customization to specific waste volumes and composition without requiring complete apparatus redesign.

The modular self-ballasting anaerobic digester apparatus 100 may include multiple technical advantages and improvements over the existing anaerobic digester apparatus. These advantages may contribute to solving the identified technical problem of creating an efficient, mobile, and self-contained anaerobic digester apparatus that can be deployed in various environments without extensive site preparation.

For instance, the modular self-ballasting anaerobic digester apparatus 100 may be operable to achieve 30% more gas output than conventional digesters due to the use of heat pipes and insulation. This increased gas production may be achieved and be related to the apparatus's 100 design and operation. For example, the heat pipes may facilitate efficient thermal energy transfer between external heat sources and the digestion environment. In an embodiment, the heat pipes may contain a working fluid with a boiling point between 30°C and 60°C, enabling effective heat transfer across the operating temperature range of the apparatus. This efficient heat transfer may enable the maintenance of optimal temperature conditions for microbial activity, potentially leading to increased biogas production.

In an embodiment, the insulation layer in the apparatus 100 may facilitate temperature stability within the digestion environment. In an embodiment, the insulation layer may be made of a high-performance synthetic material such as aramid or polyethylene terephthalate (PET) fibers. The fabric may have a thickness ranging from 2 mm to 10 mm and a density of 200 to 600 grams per square meter. This effective insulation may reduce heat loss to the environment, allowing the apparatus to maintain optimal digestion temperatures with less energy input.

In an embodiment, the combination of efficient heat transfer and effective insulation may result in more stable and favorable conditions for anaerobic microorganisms. For instance, the temperature within the digestion layer may be controlled within a range of 35°C to 55°C, depending on the specific microbial populations and desired reaction rates. This precise temperature control may promote higher metabolic activity and a more complete breakdown of organic matter, potentially leading to increased biogas yield.

In an embodiment, the modular self-ballasting anaerobic digester apparatus's 100 carbon capture and injection capabilities may further contribute to increased gas production. The ability to meter cooled carbon dioxide back into the digestion process may enable the pH levels and enhance methane production. The carbon dioxide injection rate may be modulated between 0.1 and 1.0 liters of CO₂ per cubic meter of digester volume per hour, depending on the specific conditions within the digester. The modular design of the apparatus may enable the optimization of the digestion process through the use of multiple interconnected modules. In an embodiment, a series of apparatuses may create a temperature-phased anaerobic digester process. The above-described arrangement may improve overall organic matter conversion efficiency by providing optimal conditions for different microbial populations at various stages of the digestion process.

In an embodiment, the modular self-ballasting anaerobic digester apparatus's ability to handle a wide range of organic loading rates, from 1 kg to 10 kg of volatile solids per cubic meter of digester volume per day, may contribute to its improved performance. This flexibility may enable the apparatus to be optimized for different feedstock compositions and desired biogas production rates. A 30% increase in gas output represents a significant improvement over conventional digesters, and it may be important to note that actual performance may vary depending on factors such as feedstock composition, environmental conditions, and specific operational parameters.

What is claimed is:

1. A modular self-ballasting anaerobic digestion apparatus, comprising:
a composite tanking apparatus formed within a modular structure of a mechanically connected plurality of gas-filled modules, wherein the modular structure provides a load-bearing space, a roof, a plurality of walls, and a floor;
a carbon capture apparatus operably connected to a digestion layer of the composite tanking apparatus, wherein the carbon capture apparatus, comprises:
a thermal storage tank;
a plurality of carbon capture heat pipes in the thermal storage tank, wherein the plurality of carbon capture heat pipes is operable to extract thermal energy from carbon dioxide and transfer the thermal energy to a thermal storage medium within the thermal storage tank; and
a metering valve configured to control a release of cooled carbon dioxide into the digestion layer and a pre-treatment apparatus;
a deterministic control system operably connected to a heat transfer system and the carbon capture apparatus, wherein the deterministic control system includes a plurality of sensors configured to monitor time, temperature, and liquid level parameters within the digestion layer.

2. The modular self-ballasting anaerobic digestion apparatus of claim 1, wherein the composite tanking apparatus comprises a plurality of layers, wherein the plurality of layers include:
an outer blast mitigation layer comprising a woven fabric including a plurality of ceramic spheres;
a gas-absorbent insulation layer in the outer blast mitigation layer;
a heat transfer layer comprising a sealed bladder in the gas-absorbent insulation layer; and
a digestion layer comprising a flexible bladder for storing organic matter positioned on top of the heat transfer layer.

3. The modular self-ballasting anaerobic digestion apparatus of claim 1, further comprising a plurality of heat pipes sealed within the heat transfer system, wherein the plurality of heat pipes is adapted to transfer thermal energy between a heat source and the digestion layer.

4. The modular self-ballasting anaerobic digestion apparatus of claim 1, wherein the deterministic control system includes a programmable controller configured to execute deterministic rules.

5. The modular self-ballasting anaerobic digestion apparatus of claim 1, wherein the plurality of gas-filled modules is connected using a plurality of torque tubes that are adapted to provide structural elements and guttering for liquid collection.

6. The modular self-ballasting anaerobic digestion apparatus of claim 1, wherein guttering positioned beneath a plurality of solar panels is operable to collect water runoff and direct the collected water to the digestion layer.

7. The modular self-ballasting anaerobic digestion apparatus of claim 1, wherein gas is entrapped within the plurality of gas-filled modules, providing buoyancy to create a load-bearing ballast.

8. The modular self-ballasting anaerobic digestion apparatus of claim 2, wherein the plurality of ceramic spheres in the outer blast mitigation layer includes an expandable foam coated with a ceramic liner.

9. The modular self-ballasting anaerobic digestion apparatus of claim 1, wherein the plurality of gas-filled modules is adapted to be mechanically connected to form the container structure with an aspect ratio of 5:1.

10. The modular self-ballasting anaerobic digestion apparatus of claim 1, wherein the roof includes a plurality of solar panels operable to be connected to the plurality of heat pipes.

11. The modular self-ballasting anaerobic digestion apparatus of claim 1, wherein the carbon capture system further comprises a valve controlled by the deterministic control system to meter carbon dioxide injection into the digestion layer based on the plurality of monitored parameters.

12. The modular self-ballasting anaerobic digestion apparatus of claim 1, wherein a digestion process comprises temperature-phased digestion with extended sludge retention.

13. The modular self-ballasting anaerobic digestion apparatus of claim 1, wherein a pre-treatment apparatus is operable to prepare organic matter before transferring pretreated organic matter to the digestion layer located in the load-bearing space.

14. The anaerobic digestion apparatus of claim 2, wherein the outer blast mitigation layer is configured to absorb and reduce blast effects and debris through an expansion of an expandable foam contained within the plurality of ceramic spheres.

15. The anaerobic digestion apparatus of claim 1, wherein the carbon capture apparatus is configured to achieve a reduction of at least 50% in carbon dioxide volume through thermal energy extraction via the plurality of carbon capture heat pipes.

16. The anaerobic digestion apparatus of claim 1, further comprises a pre-treatment apparatus, wherein the pre-treatment apparatus prepares organic matter before moving into the digestion layer.

17. A method of operating a modular self-ballasting anaerobic digestor apparatus, as defined in claim 1, comprising:
receiving an organic matter in the pre-treatment apparatus;
transferring a pretreated organic matter to the digestion layer within the composite tanking apparatus, wherein the composite tanking apparatus comprises a plurality of layers, including an outer blast mitigation layer, a gas-absorbent insulation layer, a heat transfer layer, and the digestion layer;
controlling temperature within the digestion layer using the plurality of carbon capture heat pipes which are sealed within the heat transfer layer;
capturing carbon dioxide produced during anaerobic digestion;
cooling the captured carbon dioxide using the plurality of carbon capture heat pipes in the thermal storage tank; and
metering the cooled carbon dioxide into the digestion layer based on a plurality of monitored parameters, including time, temperature, and liquid level within the digestion layer.
